(19) United States
(12) Patent Application Publication
(10) Pub. No.: US 2017/0356051 A1
(43) Pub. Date: Dec. 14, 2017

(54) METHOD FOR ESTIMATING SENSITIVITY TO DRUG THERAPY FOR COLORECTAL CANCER

(71) Applicants: TOHOKU UNIVERSITY, Miyagi (JP); THE UNIVERSITY OF TOKYO, Tokyo (JP)

(72) Inventors: Chikashi ISHIOKA, Miyagi (JP); Shin TAKAHASHI, Miyagi (JP); Kouta OUCHI, Miyagi (JP); Hideki SHIMODAIRA, Miyagi (JP); Hiroyuki ABURATANI, Tokyo (JP)

(21) Appl. No.: 15/518,305

(22) PCT Filed: Oct. 16, 2015
(86) PCT No.: PCT/JP2015/079909

§ 371 Date: Jul. 18, 2017

(30) Foreign Application Priority Data
Oct. 17, 2014 (JP) 2014-212503

(51) Int. Cl. C12Q 1/68

(52) U.S. Cl. CPC C12Q 1/6886; C12Q 2600/154; C12Q 2600/106

(57) ABSTRACT

The present invention relates to a method for predicting responsiveness to cancer drug therapy for colorectal cancer. More particularly, the present invention relates to a method for predicting the responsiveness of a colorectal cancer patient to cancer drug therapy, the method comprising analyzing the level of DNA methylation in a specimen comprising a colorectal cancer tissue, colorectal cancer cells, or colorectal cancer cell-derived DNA of a subject, and then determining the responsiveness of the subject to cancer drug therapy based on the level of DNA methylation.

### TECHNICAL FIELD

### Related Application

The present description includes the contents as disclosed in the description of Japanese Patent Application No. 2014-212503 (filed on Oct. 17, 2014), which is a priority document of the present application. The present invention relates to a method for predicting responsiveness to anti-cancer therapy for colorectal cancer. More particularly, the present invention relates to a method for predicting sensitivity to anti-cancer therapy for colorectal cancer, using, as an indicator, DNA methylation profiles in a specimen containing a colorectal cancer tissue, colorectal cancer cells, or colorectal cancer cell-derived DNA of a colorectal cancer patient.

### Background Art

With regard to the number of affected patients, colorectal cancer holds the second place in men and the first place in women, among all malignant tumors. With regard to the number of deaths, colorectal cancer comes in the third place (approx. 40,000 people in year 2004), and it is predicted that the number of deaths due to colorectal cancer will further increase in year 2015 (approx. 66,000 people). It is considered that the improvement of the treatment results of colorectal cancer will greatly contribute to a decrease in the number of deaths due to colorectal cancer, which accounts for 30% of a total number of deaths from cancer.

At present, metastatic colorectal cancer, which cannot be subjected to curative resection, can be treated by chemotherapies based on an irinotecan and based on oxaliplatin. The order in which these agents are applied in the combined use thereof has not been particularly studied.

On the other hand, as a result of the introduction of molecular targeted drugs, and in particular, the introduction of anti-EGFR antibody drugs (cetuximab and panitumumab) and an anti-VEGF antibody drug (bevacizumab), the treatment results (progression-free survival and overall survival) of metastatic colorectal cancer have been steadily improved. However, such molecular targeted drugs are expensive, and at the present moment, the cost-effectiveness of the molecular targeted drugs is inferior to that of conventional chemotherapeutic agents or molecular targeted drugs used for other cancers. From the viewpoint of avoiding the side effects of invalid patients that would cause unnecessary health care costs, it is necessary to selectively apply treatments to more effective subjects.

With regard to a biomarker for predicting the therapeutic sensitivity of metastatic colorectal cancer to anti-EGFR antibody drugs, it has been reported in 2008 that anti-EGFR antibody drugs do not increase therapeutic effects in the case of having a mutation on exon 2 of KRAS. Moreover, in clinical studies conducted in recent years, it has been reported that the effects of anti-EGFR antibody drugs are further increased in the case of wild-type RAS that does not have mutations on exons 3 and 4 as well as exon 2 of KRAS and exons 2, 3 and 4 of NRAS. Furthermore, a PIK3CA mutation is promising as a therapeutic effect predicting factor, and further, a BRAF mutation has been reported as a prognosis predicting factor, so far.

However, in the case of wild-type exon 2 of KRAS, which is a widely used biomarker at present, an increase in the response rate by the use of an anti-EGFR antibody drug is merely about 30%, and this is not considered to be sufficient. Even taking into consideration the aforementioned other genetic mutations, it is considered difficult to identify an authentic sensitivity group only by an analysis based on genetic mutation.

In contrast, Aburatani et al. have reported a method which comprises analyzing the methylation state of a marker gene in DNA extracted from a biological sample, and then determining the presence or absence of cancer cells in the biological sample or the prognosis of a colorectal cancer patient based on the obtained results (Patent Literature 1). Moreover, Yagi et al. have reported that when HME (a highly-methylated group) is extracted based on the methylation state of a first gene group, and IME (an intermediately-methylated group) and LME (a low-methylated group) are then extracted based on the methylation state of a second gene group, and thus, when a colorectal cancer patient group is classified into three subtypes, the survival period of IME (including a KRAS gene mutation) is found to be shortest (Non Patent Literature 1).

As a method for enabling a selective treatment of colorectal cancer, Ishioka et al. have reported a method which comprises comprehensively analyzing the expression levels of genes in colorectal cancer tissues, and attributing the results to any one of previously classified four groups, so as to predict the responsiveness of a colorectal cancer patient to an anti-EGFR antibody (Patent Literature 2).

A group from Sapporo Medical University has reported that the methylation level of LINE-1 is positively correlated with the expression level of microRNA-31 in a colorectal cancer patient, and that with regard to progression-free survival in cases of administration of an anti-EGFR antibody drug, the progression-free survival in a microRNA-31 high expression group is significantly shorter than that in a low expression group (Non Patent Literature 2).

Furthermore, Lee et al. have proposed a hypothesis that the DNA methylation of a CpG island would be associated with the biological properties of cancer, and that sensitivity to an anti-EGFR antibody would be influenced by the methylation state of DNA (Non Patent Literature 3).

### CITATION LIST

### Patent Literature

Patent Literature 1: Japanese Patent Laid-Open No. 2013-183725
Patent Literature 2: WO 2011/002029

### Non Patent Literature

Non Patent Literature 1: Yagi K. et al., Clin Cancer Res. 2010 Jan. 1; 16(1): 21-33
Non Patent Literature 2: Katsuhiko NOSHO, Daiwa Securities Health Foundation, Year 2012, (39th) Search Study Subsidy Report
Non Patent Literature 3: Michael Sangmin Lee et al., ASCO Annual Meeting 2014, Abstract Number 3533 (http://meetinglibrary.asco.org/content/134359-144)

### SUMMARY OF INVENTION

### Technical Problem

In guidelines for administration of an anti-EGFR antibody used as a therapeutic agent for metastatic colorectal cancer, a method of administering the present antibody only to patients having a wild-type KRAS gene has been recommended. However, there are not a few cases where even wild-type KRAS gene patients show resistance to the anti-EGFR antibody. Hence, administration of the expensive anti-EGFR antibody to patients who are resistant to the present antibody causes high economical and/or physical burdens on the patients, and thus, it has been desired to develop guidelines for administration of the anti-EGFR antibody, which provide higher cost-effectiveness to the patients.

The present invention has been made under the aforementioned circumstances. It is an object of the present invention to predict with high precision the responsiveness of colorectal cancer to anti-cancer therapy, to reduce economical and/or physical burdens on patients, and to provide administration guidelines causing higher cost-effectiveness.

### Solution to Problem

The present inventors have comprehensively analyzed the level of DNA methylation in the tissues from colorectal cancer patients. As a result, the inventors have found that the treatment results of anti-cancer therapy on a low-methylated group are significantly higher than those on a highly-methylated group, thereby completing the present invention.

Specifically, the present invention provides the following [1] to [14].

[1] A method for predicting the responsiveness of a colorectal cancer patient to cancer drug therapy, the method comprising analyzing a level of DNA methylation in a specimen comprising a colorectal cancer tissue, colorectal cancer cells, or colorectal cancer cell-derived DNA of a subject, and then determining the responsiveness of the subject to cancer drug therapy based on the level of DNA methylation;
[2] the method according to the above [1], which comprises the following steps:
(1) a step of measuring a level of DNA methylation in a specimen comprising a colorectal cancer tissue, colorectal cancer cells, or colorectal cancer cell-derived DNA of a subject,
(2) a step of defining a gene having a β value of 0.5 or more as methylation positive, and then, classifying the subject into a highly-methylated group when the ratio of a methylation-positive gene is 60% or more, and classifying the subject into a low-methylated group when the ratio of a methylation-positive gene is less than 60%, and
(3) a step of determining that the subject is sensitive to cancer drug therapy when the subject is classified into the low-methylated group, and determining that the subject is resistant to cancer drug therapy when the subject is classified into the highly-methylated group;
[3] the method according to the above [1] or [2], wherein the analysis is carried out on at least 4 or more marker genes, as targets, selected from a group of genes having a significant difference in the β value between the highly-methylated group and the low-methylated group;
[4] the method according to the above [1] or [2], wherein the analysis is carried out on at least 4 or more marker genes, as targets, selected from a group of genes shown in Table 7 or a group of genes shown in Table 8, and for example, the method according to the above [1] or [2], wherein the analysis is carried out on the group of genes shown in Table 8 as targets;
[5] the method according to the above [1] or [2], wherein the analysis is carried out on 4 to 20 marker genes, as targets, selected from a group of genes shown in Table 7 or a group of genes shown in Table 8;
[6] the method according to the above [1] or [2], wherein the analysis is carried out on 4 to 10 marker genes, as targets, selected from a group of genes shown in Table 7 or a group of genes shown in Table 8;
[7] the method according to any one of the above [4] to [6], wherein the marker genes include at least one or more gene selected from the 24 genes shown in Table 8, or CACNA1G, LOX, SLC30A10, ELMO1, HAND1, IBN2 and THBD;
[8] the method according to any one of the above [1] to [7], wherein the cancer drug therapy is chemotherapy;
[9] the method according to any one of the above [1] to [7], wherein the cancer drug therapy is a therapy using a molecular targeted drug;
[10] the method according to the above [9], wherein the molecular targeted drug is an anti-EGFR antibody;
[11] the method according to any one of the above [1] to [10], wherein the suitability of the order of cancer drug therapies can be determined;
[12] a probe set for predicting the responsiveness of a colorectal cancer patient to cancer drug therapy, wherein

the probe set comprises a probe which comprises a sequence complementary to a region comprising a CpG site of at least one of, 4 or more marker genes selected from a group of genes shown in Table 7 or a group of genes shown in Table 8, and for example, all the genes in the group of genes shown in Table 8, and which is capable of detecting the presence or absence of the methylation of the CpG site;

[13] the probe set according to the above [12], wherein the marker genes comprise one or more genes selected from the group of genes shown in Table 8, or CACNA1G, LOX, SLC30A10, ELMO1, HAND1, IBN2 and THBD;
[14] a kit for predicting the responsiveness of a colorectal cancer patient to cancer drug therapy, wherein the kit comprises:
(a) a probe which comprises a sequence complementary to a region comprising a CpG site of at least one of, 4 or more genes selected from a group of genes shown in Table 7 or a group of genes shown in Table 8, and for example, all the genes in the group of genes shown in Table 8, and which is capable of detecting the presence or absence of the methylation of the CpG site, and
(b) a primer pair which binds to a region comprising a CpG site of at least one of, 4 or more genes selected from a group of genes shown in Table 7 or a group of genes shown in Table 8, and for example, all the genes in the group of genes shown in Table 8, and which is capable of amplifying the region comprising the CpG site; and
[15] the kit according to the above [14], wherein the marker genes comprise one or more genes selected from the 24 genes shown in Table 8, or CACNA1G, LOX, SLC30A10, ELMO1, HAND1, IBN2 and THBD.

To date, phenotype classification based on methylation profiles including CIMP as a typical example has been reported with regard to colorectal cancer or several other cancers. However, the correlation of drug sensitivity with methylation has not yet been reported, and thus, it is not easy to predict from previous reports the presence of the correlation of methylation profiles with drug sensitivity. That is to say, the present invention is a first report regarding possible prediction of drug sensitivity from methylation profiles.

### Advantageous Effects of Invention

According to the present invention, it becomes possible to select chemotherapy for colorectal cancer, and particularly, unresectable metastatic colorectal cancer, based on a difference in methylation state. Specifically, when a primary treatment is started, with regard to the regimens of irinotecan-based and oxaliplatin-based chemotherapies, both of which are considered to be favorable at present, the order in which the regimens are applied can be selected based on the DNA methylation state of a specimen derived from a patient.

In addition, according to the present invention, a case group, which shows resistance to an anti-EGFR antibody drug even if it is wild-type KRAS, can be extracted. Moreover, even the recently reported wild-type RAS cases having no mutations on exons 3 and 4 as well as exon 2 of KRAS, and on exons 2, 3 and 4 of NRAS, which are included in a treatment-resistant group, can be extracted. That is to say, the method of the present invention can extract actually resistant cases from cases that have been classified into a treatment-sensitive group according to conventional reports, and thus, the present method is considered to be a method for predicting therapeutic effects with higher precision.

Genetic mutations are successively accumulated in the onset and/or progression of a cancer, and subpopulations having various genetic mutation profiles are present in tumor (heterogeneity). In the case of colorectal cancer, accumulation of genetic mutations is highly likely to occur in the onset and/or progression of a tumor, and colorectal cancer is a tumor rich in heterogeneity. Accordingly, when genetic mutations are to be examined in colorectal cancer, the results are strongly influenced by at what time point in the therapeutic process, from what site, from what range of tumor, DNA was extracted.

In contrast, it is considered that methylation profiles are determined in the initial stage of canceration, and thus, it can be said that the methylation profiles are relatively uniform in a tumor. That is, it is expected that, when compared with a diagnosis based on genetic mutation, the method of the present invention suppresses a variation in the results caused by the aforementioned specimen collection conditions, and also more precisely reflects methylation profiles in a tumor at the start of use of a molecular targeted drug, even if it is a specimen collected upon resection of a primary lesion. Specifically, the method of the present invention can precisely determine the therapeutic effects of cancer drug therapy, regardless of the state of progress of cancer, or specimen collection conditions.

Furthermore, since the method of the present invention can concentrate a group in which the effects of an anti-EGFR antibody are high, and then can conduct detection, when compared with conventional methods based on gene expression, the present method can conduct higher-precision determination than conventional methods, even in therapeutic methods of using molecular targeted drugs.

### BRIEF DESCRIPTION OF DRAWINGS

FIG. 1 shows the results of the comprehensive DNA methylation analysis of 45 colorectal cancer cases having usage history of an anti-EGFR antibody drug (unsupervised hierarchical cluster analysis using 3163 probes having a standard deviation in β value distribution of greater than 0.25).

FIG. 2 shows a comparison between a highly-methylated group and a low-methylated group, in terms of (A) progression-free survival (PFS) and (B) overall survival (OS) when an anti-EGFR antibody drug has been used for the 45 colorectal cancer cases.

FIG. 3 shows the results of the comprehensive DNA methylation analysis of 52 colorectal cancer cases having usage history of an anti-EGFR antibody drug, which are different from the 45 cases in Example 1 (unsupervised hierarchical cluster analysis using 2577 probes having a standard deviation in β value distribution of greater than 0.25).

FIG. 4 shows a comparison between a highly-methylated group and a low-methylated group, in terms of (A) progression-free survival (PFS) and (B) overall survival (OS) when an anti-EGFR antibody drug has been used for the 52 colorectal cancer cases.

FIG. 5 shows the progression-free survival (PFS) upon the use of an anti-EGFR antibody drug: (A) a comparison between a highly-methylated group and a low-methylated group according to the present classification, and (B) a comparison between a mutant RAS group and a wild-type RAS group.

FIG. 6 shows the overall survival (OS) after completion of the initial administration of an anti-EGFR antibody drug: (A) a comparison between a highly-methylated group and a low-methylated group according to the present classification, and (B) a comparison between a mutant RAS group and a wild-type RAS group.

FIG. 7 shows the survival curve upon the use of an anti-EGFR antibody drug: (A) a comparison between a highly-methylated group and a low-methylated group according to the present classification, (B) a comparison among a highly-methylated group (HME), an intermediately-methylated group (IME) and a low-methylated group (LME) based on the classification of Yagi et al.

FIG. 8 shows the correlation of the progression-free survival (PFS) when a combination therapy comprising oxaliplatin (solid line) or a combination therapy comprising irinotecan (broken line) has been performed as a primary treatment on metastatic colorectal cancer, with methylation classification: (A) the results of the primary treatment of a highly-methylated (HMCC) group, and (B) the results of the primary treatment of a low-methylated (LMCC) group.

FIG. 9 shows the correlation of the progression-free survival (PFS) when a combination therapy comprising oxaliplatin (solid line) or a combination therapy comprising irinotecan (broken line) has been performed as a secondary treatment on metastatic colorectal cancer, with methylation classification: (A) the results of the secondary treatment of a highly-methylated (HMCC) group, and (B) the results of the secondary treatment of a low-methylated (LMCC) group.

FIG. 10 shows the correlation of the progression-free survival (PFS) when a combination therapy comprising oxaliplatin as a primary treatment and also comprising irinotecan as a secondary treatment (solid line), or a combination therapy comprising irinotecan as a primary treatment and also comprising oxaliplatin as a secondary treatment (broken line) has been performed on metastatic colorectal cancer, with methylation classification: (A) the treatment results of a highly-methylated (HMCC) group, and (B) the treatment results of a low-methylated (LMCC) group.

FIG. 11 shows the correlation of the overall survival (OS) when a combination therapy comprising oxaliplatin as a primary treatment and also comprising irinotecan as a secondary treatment (solid line), or a combination therapy comprising irinotecan as a primary treatment and also comprising oxaliplatin as a secondary treatment (broken line) has been performed on metastatic colorectal cancer, with methylation classification: (A) the treatment results of a highly-methylated (HMCC) group, and (B) the treatment results of a low-methylated (LMCC) group.

FIG. 12 shows the correlation of the progression-free survival (PFS) when a combination therapy comprising oxaliplatin (solid line) or a combination therapy comprising irinotecan (broken line) has been performed as a primary treatment on metastatic colorectal cancer, with CIMP classification: (A) the results of the primary treatment of a CIMP-positive group, and (B) the results of the primary treatment of a CIMP-negative group.

FIG. 13 shows the correlation of the progression-free survival (PFS) when a combination therapy comprising oxaliplatin (solid line) or a combination therapy comprising irinotecan (broken line) has been performed as a secondary treatment on metastatic colorectal cancer, with CIMP classification: (A) the results of the secondary treatment of a CIMP-positive group, and (B) the results of the secondary treatment of a CIMP-negative group.

FIG. 14 shows the correlation of the progression-free survival (PFS) when a combination therapy comprising oxaliplatin as a primary treatment and also comprising irinotecan as a secondary treatment (solid line), or a combination therapy comprising irinotecan as a primary treatment and also comprising oxaliplatin as a secondary treatment (broken line) has been performed on metastatic colorectal cancer, with CIMP classification: (A) the results of the primary treatment of a CIMP-positive group, and (B) the results of the primary treatment of a CIMP-negative group.

FIG. 15 shows the correlation of the overall survival (OS) with CIMP classification, when a combination therapy comprising oxaliplatin as a primary treatment and also comprising irinotecan as a secondary therapy (solid line), or a combination therapy comprising irinotecan as a primary treatment and also comprising oxaliplatin as a secondary treatment (broken line) has been performed on metastatic colorectal cancer: (A) the results of the primary treatment (oxaliplatin) of a CIMP-positive group, (B) the results of secondary treatment (oxaliplatin) of a CIMP-positive group, (C) the results of the primary/secondary therapies (oxaliplatin/irinotecan) of a CIMP-positive group, (D) the results of primary treatment (oxaliplatin) of a CIMP-negative group, (E) the results of the secondary treatment (oxaliplatin) of a CIMP-negative group, and (F) the results of primary/secondary therapies (oxaliplatin/irinotecan) of a CIMP-negative group.

FIG. 16 shows the procedures for narrowing probes in two cohorts and verification thereof (Example 7).

FIG. 17 shows the results obtained by classifying again 97 cases as analysis targets into an HMCC group and an LMCC group, using 24 markers (probes) that have been narrowed up by the analysis of the two cohorts. In the figure, the columns indicate individual cases (97 columns in total), and the red or blue color in the uppermost row shows that each case is classified into either HMCC or LMCC by the first analysis using 3144 or 2577 probes. The lines in the second row and so on (24 lines in total) indicate individual probes. The orange color shows that it has been determined that the case is methylation positive (=β value of 0.5 or greater), and the green color shows that it has been determined that the case is methylation negative (=β value of less than 0.5).

### DESCRIPTION OF EMBODIMENTS

### 1. Definition

The present invention relates to a method for determining the responsiveness of a colorectal cancer patient to cancer drug therapy. Hereafter, the meanings of the terms used in the present invention and in the present description will be described.

In the present invention, the term “colorectal cancer” means a carcinoma developed in the large intestine (cecum, colon, and rectum), which includes carcinomas developed in the anal canal. The term “colorectal cancer patient” includes a subject who is suspected of having colorectal cancer and needs to examine the responsiveness to cancer drug therapy, as well as a subject who is affected with colorectal cancer.

The “cancer drug therapy” is not particularly limited, and examples of the cancer drug therapy include both a chemotherapy of using oxaliplatin, irinotecan and the like, and a therapy of using molecular targeted drugs such as an anti-EGFR antibody.

In the present invention, the term “anti-EGFR antibody” is used to mean an antibody specific to EGFR (epidermal growth factor receptor), or an immunologically active fragment thereof. Examples of such an anti-EGFR antibody include cetuximab that is a commercially available IgG 1 subclass human-mouse chimeric antibody, panitumumab that is an IgG 2 subclass completely human antibody, and further, all of anti-EGFR antibodies that are useful as molecular targeted drugs for cancer.

Approximately 80% of metastatic and/or recurrent colorectal cancers express EGFR, and the growth of cancer cells is suppressed by inhibiting the EGFR located most upstream of signaling with antibodies. However, there are cases where signaling is not inhibited even if the EGFR is blocked by antibodies. For example, as described above, it has been known that in the case of a patient having a mutation in K-RAS located downstream of a growth signaling pathway, signaling is not inhibited even by blocking EGFR.

In the present invention, the phrase “responsiveness to cancer drug therapy” means the responsiveness of a patient to cancer drug therapy, as described above. When the cancer drug therapy has effects on the patient, the patient is indicated to be “sensitive” to the therapy, and when the cancer drug therapy does not have effects on the patient, the patient is indicated to be “resistant” to the therapy.

The “specimen” used in the present invention is not particularly limited, as long as it contains a suspected lesion area isolated from a subject, namely, colorectal cancer cell-derived DNA (e.g., DNA derived from tumor in the plasma), such as colorectal cancer tissues or colorectal cancer cells.

The “DNA methylation” may occur at the carbon atom at position 5 of the pyrimidine ring of cytosine constituting DNA, or at the nitrogen atom at position 6 of the purine ring of adenine constituting DNA. In general, in the somatic tissues of an adult mammal, such DNA methylation occurs in a CpG site (i.e., a dinucleotide site in which cytosine is adjacent to guanine). In the case of cancer, hypermethylation is frequently observed in the CpG site, and particularly, in the CpG island in the promoter region. On the other hand, hypomethylation is also associated with progression of cancer.

The “DNA methylation” according to the present invention is not limited to the methylation of a CpG site, and it includes methylation of non-CpG sites, such as methylation regions in non-CpG sites known in human stem cells, and regions exhibiting different methylation between known normal cells and cancer cells.

The “level of DNA methylation” according to the present invention means the ratio of methylation (methylation/methylation+non-methylation), and for example, it is indicated with a β value. It is to be noted that such a β value is calculated by the following formula:

β value=(maximum value of fluorescence values of methylation-detecting probes)/(maximum value of fluorescence values of non-methylation-detecting probes+maximum value of fluorescence values of methylation-detecting probes+100)

The “marker gene” used to measure the level of DNA methylation is not particularly limited. All genes contained in a specimen may be used as targets and may be comprehensively analyzed, or the targets may be limited to specific genes, and the specific genes may be then analyzed. The marker genes are preferably 4 or more genes selected from a group of genes having a significant difference in the β value between a highly-methylated group and a low-methylated group, and specifically, the present marker genes are selected from a group of 1,053 genes shown in Table 7 or a group of 24 genes shown in Table 8. For example, the marker genes include genes selected from the 7 genes indicated with the genetic symbols CACNA1G, LOX, SLC30A10, ELMO1, HAND1, IBN2 and THBD, or the 24 genes which are specified with the chromosome numbers and location information shown in Table 8.

### 2. Method for Determining Responsiveness to Cancer Drug Therapy

In the present invention, the responsiveness of a colorectal cancer patient to cancer drug therapy is determined based on the level of DNA methylation in a specimen containing a colorectal cancer tissue or colorectal cancer cells of the aforementioned patient.

The method of the present invention comprises, for example, the following steps:

(1) a step of measuring the level of DNA methylation in a specimen containing a colorectal cancer tissue, colorectal cancer cells, or colorectal cancer cell-derived DNA of a subject (measurement step),
(2) a step of defining a gene having a β value of 0.5 or more as methylation positive, and then, classifying the subject into a highly-methylated group when the ratio of a methylation-positive gene is 60% or more, and classifying the subject into a low-methylated group when the ratio of a methylation-positive gene is less than 60% (analysis and/or classification step), and
(3) a step of determining that the subject is sensitive to cancer drug therapy when the subject is classified into the low-methylated group, and determining that the subject is resistant to cancer drug therapy when the subject is classified into the highly-methylated group (determination step).

### 2.1 Measurement Step

### (1) Extraction of DNA

First, genomic DNA is extracted from a specimen isolated from a subject. DNA extraction may be carried out according to a method known in the present technical field. Such DNA extraction can be carried out, for example, using a commercially available kit (QIAamp DNA Micro Kit (QIAGEN), NucleoSpinR Tissue (TAKARA), etc.).

### (2) Measurement of the Level of DNA Methylation

The measurement of the level of DNA methylation is not particularly limited, and examples of the measurement method include (A) an analysis method involving a bisulfite treatment and sequencing, (B) a method comprising fragmenting methylated DNA, concentrating it, and then analyzing the methylated DNA, (C) an analysis method of utilizing methylation-sensitive restriction enzymes, and (D) an analysis method of utilizing a methylation-specific PCR method. All of these methods may be applied.

As a preferred example, there is a method of using the bead arrays of Illumina (Infinium (registered trademark) Human Methylation 450 BeadChip). In this method, cytosine that has not been methylated in DNA (non-methylated cytosine) is converted to uracil by performing a bisulfite treatment, so that the methylated cytosine can be distinguished from the non-methylated cytosine. Thereafter, probes immobilized on two beads, namely, a methylation probe (type M) and a non-methylation probe (type U), which are specific to individual sites, are hybridized, and a single nucleotide elongation reaction is then carried out using labeled ddNTP, so that the ratio between methylation and non-methylation is calculated based on these fluorescence intensity signals. Thereby, a comprehensive DNA methylation analysis can be simply carried out.

Another example can be the MassARRAY method of Sequenom. In this method, DNA methylation is analyzed by utilizing a difference in masses caused by a difference in the nucleotide sequences of regions to be analyzed. Specifically, non-methylated cytosine is converted to uracil by treating DNA with bisulfite (wherein methylated cytosine is not converted), and the presence or absence of methylation is then analyzed based on a difference in the masses of the nucleotides G and A in the complementary strand thereof. Thereby, large quantities of samples can be quantitatively analyzed in a short time.

The level of DNA methylation may be measured for all genes contained in a specimen. However, the present inventors have found that the responsiveness of a subject to cancer drug therapy can be determined by measuring the methylation levels of specific genes. Such specific genes are 4 or more genes selected from a group of genes having a significant difference in the β value between a highly-methylated group and a low-methylated group, and specifically, such specific genes are selected from a group of 1,053 genes shown in Table 7 or a group of 24 genes shown in Table 8. For example, the marker genes include genes selected from the 7 genes indicated with the genetic symbols CACNA1G, LOX, SLC30A10, ELMO1, HAND1, IBN2 and THBD. Otherwise, the marker genes include genes selected from the 24 genes specified with the chromosome numbers and location information shown in Table 8.

By analyzing the methylation levels of 4 or more genes, preferably 4 to 7 genes, more preferably 4 to 20 genes, and further preferably 4 to 10 genes selected from the above described marker genes, the responsiveness of a subject to cancer drug therapy can be predicted.

2.2 Analysis and/or Classification Step

### (1) Analysis of the Level of DNA Methylation

Subsequently, the above described measurement results are analyzed, and the subject is classified into either a highly-methylated group or a low-methylated group. The level of DNA methylation can be quantified, for example, using the aforementioned β value or the like. This β value is calculated and/or analyzed for all genes or the above described specific genes, so that the subject can be classified into either a highly-methylated group or a low-methylated group.

(2) Classification into Highly-Methylated Group or Low-Methylated Group

The subject may be classified into a highly-methylated group or a low-methylated group by performing the comparative analysis of the results of the subject with the profiles of the level of DNA methylation in a specimen that has been previously obtained from a colorectal cancer patient, or the subject may also be classified based on a predetermined cut-off value experimentally determined from accumulated data.

As described in the Examples of the present application, the present inventors have found that, when the aforementioned marker gene having a β value of 0.5 or more is defined as methylation positive, and in a case where the ratio of a methylation-positive gene is 60% or more, the subject can be classified into a highly-methylated group, and in a case where the ratio of a methylation-positive gene is less than 60%, the subject can be classified into a low-methylated group. According to this method, the subject can be simply classified into a highly-methylated group or a low-methylated group, based on the methylation levels of at least 4 marker genes.

### 2.3 Determination Step

Based on the above described classification results, when the subject is classified into a low-methylated group, it is determined that the subject is sensitive to cancer drug therapy, whereas when the subject classified into a highly-methylated group, it is determined that the subject is resistant to cancer drug therapy.

### 2.4 Application to Selection of Treatment

The method of the present invention can be applied to selection of chemotherapy for colorectal cancer, and in particular, for unresectable metastatic colorectal cancer, based on a difference in methylation states. That is to say, when a primary treatment is initiated, it is considered at present that both the regimen of an irinotecan-based chemotherapy and the regimen of an oxaliplatin-based chemotherapy may be available. However, by using the method of the present invention, it can be determined that a patient in a highly-methylated group should receive an irinotecan-based chemotherapy, and on the other hand, it can be determined that a patient in a highly-methylated group who has initiated to receive an irinotecan-based chemotherapy should receive an oxaliplatin-based chemotherapy as a secondary treatment. On the other hand, it can be determined that a patient in a low-methylated group may receive either an irinotecan-based chemotherapy or an oxaliplatin-based chemotherapy as a primary treatment.

In the method of the present invention, from among cases which have been classified into a treatment-sensitive group according to the previous reports, actually resistant cases can be extracted, so that it becomes possible to predict therapeutic effects with higher precision. Moreover, regardless of the state of progress of cancer or conditions for specimen collection, therapeutic effects can be precisely determined not only regarding chemotherapy, but also regarding therapies of using molecular targeted drugs such as an anti-EGFR antibody.

Furthermore, in the method of the present invention, a lower p value is found between a treatment-sensitive group and a treatment-resistant group, than in the case of classification based on expression arrays, and it is possible to concentrate a group having high therapeutic effects, so that determination can be carried out with higher precision.

Further, as described in the after-mentioned Examples, in the method of the present invention, two independent case groups having a significant difference in terms of response rate, progression-free survival (PFS: Progression-Free Survival), and overall survival (OS: Overall Survival) upon the use of an anti-EGFR antibody drug have been successfully extracted, and it has also been demonstrated that the present method is excellent in terms of reproducibility.

Regarding the guidelines for administration of an anti-EGFR antibody used as a therapeutic agent for metastatic colorectal cancer, a method of administering the present antibody only to patients with a wild-type KRAS gene has been recommended. The method of the present invention is based on an epigenetic method that is different from conventional genetic methods, and the present method is basically different from conventional methods in that the present method enables the extraction of patients having resistance to the present antibody from a group of patients who have been classified to be sensitive to the present antibody under the current administration guidelines.

### 3. Kit and/or Probe Set for Predicting Responsiveness to Cancer Drug Therapy

The present invention also provides a probe set and a kit for predicting the responsiveness of a colorectal cancer patient to cancer drug therapy.

The probe set of the present invention comprises a probe which comprises a sequence complementary to a region comprising the CpG site of at least one of 4 or more marker genes selected from the gene group shown in Table 7 or Table 8, and which is capable of detecting the presence or absence of the methylation of the CpG site. Herein, the presence or absence of methylation means a probe capable of detecting the cytosine in a methylation site and the uracil in a non-methylation site, in the case of bisulfite sequencing. It is to be noted that the above described marker genes preferably include one or more genes selected from CACNA1G, LOX, SLC30A10, ELMO1, HAND1, IBN2 and THBD.

The kit of the present invention comprises

(a) a probe which comprises a sequence complementary to a region comprising the CpG site of at least one of 4 or more marker genes selected from the gene group shown in Table 7 or Table 8, and which is capable of detecting the presence or absence of the methylation of the CpG site, and
(b) a primer pair which binds to a region comprising the CpG site of at least one of 4 or more marker genes selected from the gene group shown in Table 7 or Table 8, and which is capable of amplifying the region comprising the CpG region.

It is to be noted that the above described marker genes preferably include one or more genes selected from the genetic symbols CACNA1G, LOX, SLC30A10, ELMO1, HAND1, IBN2 and THBD. Otherwise, the marker genes preferably include genes selected from the 24 genes specified with the chromosome numbers and location information shown in Table 8.

By using the probe set or kit of the present invention, the responsiveness of a colorectal cancer patient to cancer drug therapy can be simply and highly precisely predicted.

As described in the after-mentioned Examples, both the method based on expression arrays and the method of the present invention use comprehensive data to carry out an unsupervised hierarchical cluster analysis, thereby identifying subgroups having different drug sensitivity. However, the method of the present invention that is based on methylation analysis is considered to be a more practical invention because it has succeeded in identifying several probe sets capable of extracting two groups having a significant difference in drug sensitivity.

### EXAMPLES

Hereinafter, the present invention will be described in more detail in the following Examples. However these Examples are not intended to limit the scope of the present invention.

### Example 1: Comprehensive DNA Methylation Analysis of Using 45 Colorectal Cancer Cases

Using formalin-fixed paraffin-embedded tissues (FFPE specimen) of colorectal cancer tumor tissues that had been surgically excised from 45 colorectal cancer cases having usage history of an anti-EGFR antibody drug, a comprehensive DNA methylation analysis was carried out by employing Infinium 450K (Illumina). It is to be noted that the target cases were set to be cases in which no mutations were found in KRAS exon 2 according to a Sanger method.

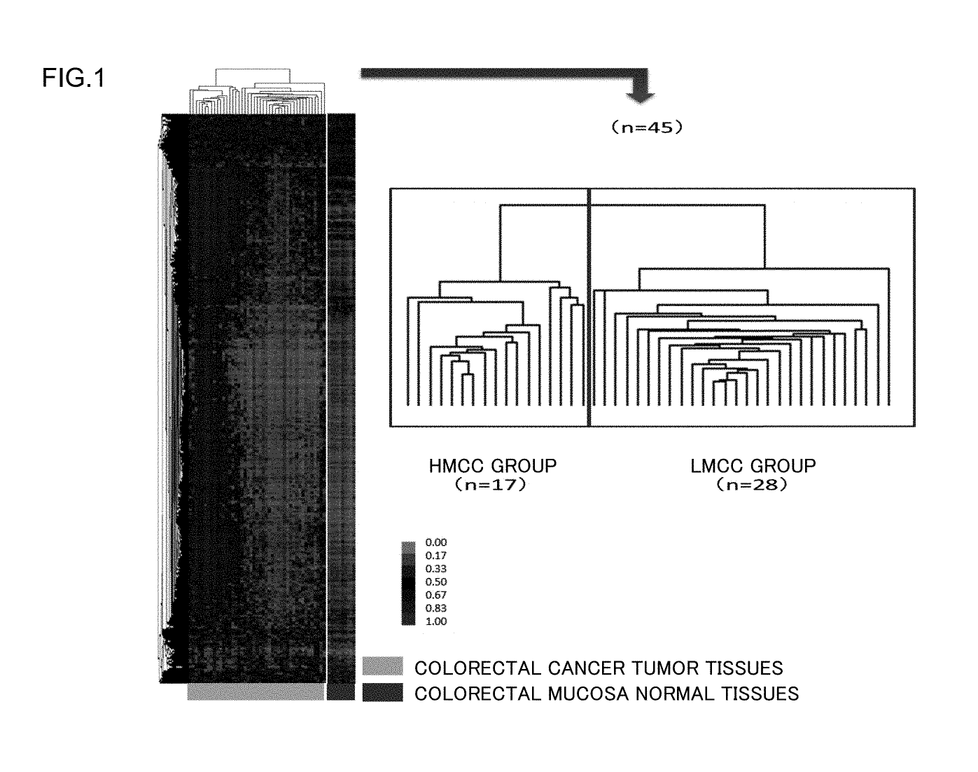

The β value of each probe (methylated probes/methylated probes+non-methylated probes) was calculated, and thereafter, 3,163 probes having a standard deviation in the β value distribution that was greater than 0.25 were used to carry out an unsupervised hierarchical cluster analysis (FIG. 1).

As a result of the above described analysis, the analysis target cases were classified into two groups, namely, a Highly-Methylated Colorectal Cancer (HMCC) group (17 cases) having a high methylation level and a Low-Methylated Colorectal Cancer (LMCC) group (28 cases) having a low methylation level.

The response rate to a high EGFR antibody drug was compared between the above described two groups (HMCC group and LMCC group) (Table 1). When the response rate to the anti-EGFR antibody drug has been focused, the response rate of the LMCC group was found to be 36% (10 cases), whereas the response rate of the HMCC group was found to be 6% (1 case). Thus, the response rate of the LMCC group was significantly high (p=0.03).

TABLE 1
Comparison of response rate to anti-EGFR antibody
drug in Cohort 1 between two groups

| | All samples | | HMCC group | | LMCC group | | |
|---|---|---|---|---|---|---|---|
| | Number of | | Number of | | Number of | | p |
| | subjects | % | subjects | % | subjects | % | value |
| RR (%) | | 25.0 | | 6.3 | | 35.7 | 0.03 |
| CR | 0 | 0 | 0 | 0 | 0 | 0 | |
| PR | 11 | 25.0 | 1 | 6.3 | 10 | 35.7 | |
| SD | 15 | 34.1 | 4 | 25.0 | 11 | 39.3 | |
| PD | 18 | 40.9 | 11 | 68.8 | 7 | 25.0 | |
| NA | 1 | | 1 | | 0 | | |

CR: complete response,
PR: partial response,
SD: stable,
PD: progressed,
RR: response rate

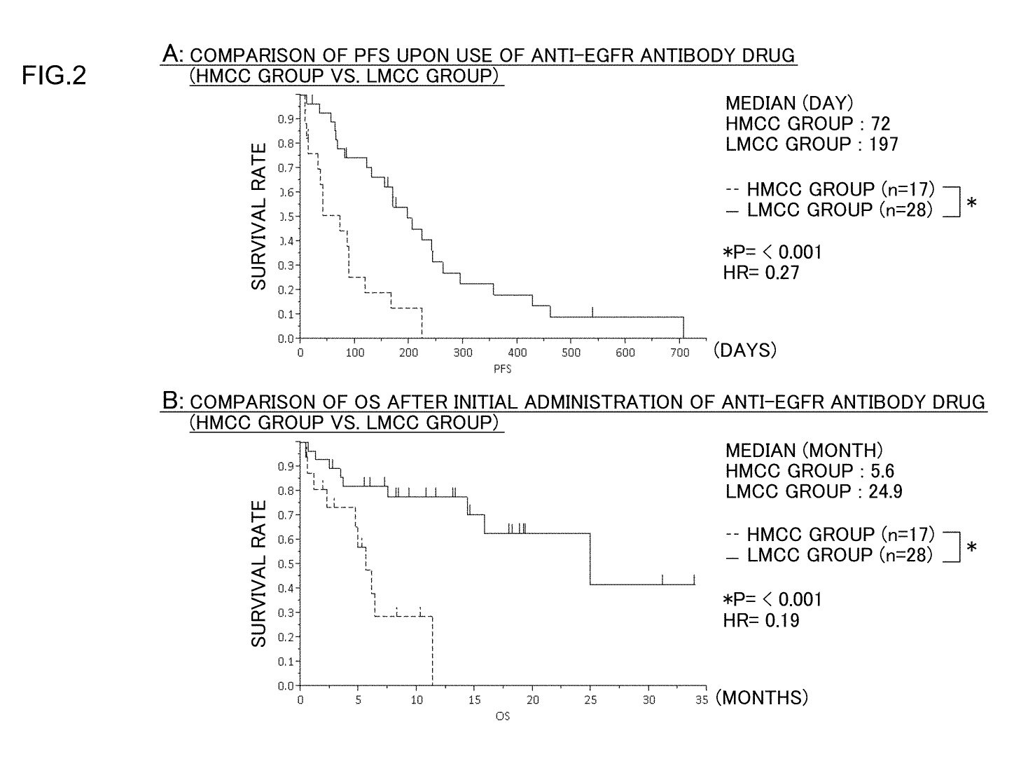

When the progression-free survival (PFS) upon the use of an anti-EGFR antibody drug was focused, the median of the LMCC group was found to be 197 days, whereas the median of the HMCC group was found to be 72 days. Thus, the progression-free survival (PFS) was significantly prolonged in the LMCC group (p≦0.001, HR=0.27: FIG. 2A).

With regard to a comparison in terms of the overall survival (OS) after completion of the initial administration of an anti-EGFR antibody drug, the median of the LMCC group was found to be 24.9 months, whereas the median of the HMCC group was found to be 5.6 months. Thus, the overall survival (OS) was significantly prolonged in the LMCC group (p≦0.001, HR=0.19: FIG. 2B).

From the aforementioned results, a significant difference was found in all of response rate, PFS, and OS, upon the use of an anti-EGFR antibody drug, between the two groups that had been classified based on the comprehensive DNA methylation analysis, and thus, it was strongly suggested that therapeutic effects could be predicted.

### Example 2: Verification of Independent 52 Colorectal Cancer Cases

Using 52 colorectal cancer cases having usage history of an anti-EGFR antibody drug, which were independent from the 45 cases used in Example 1, a comprehensive DNA methylation analysis was carried out by employing Infinium 450K. As with Example 1, the target cases were set to be cases in which no mutations were found in KRAS exon 2 according to a Sanger method.

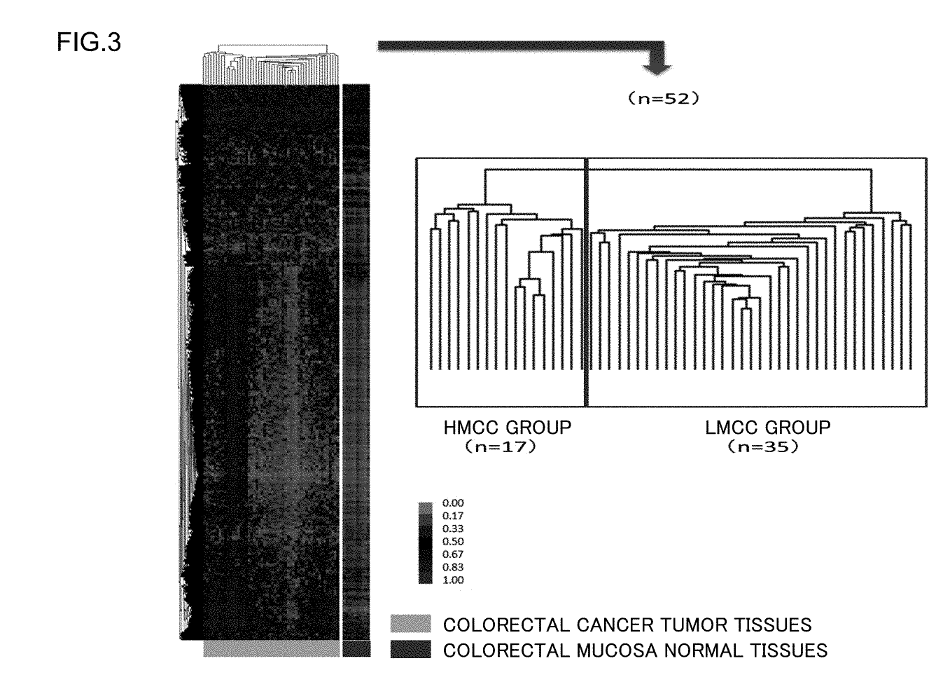

As with Example 1, the β value of each probe (methylated probes/methylated probes+non-methylated probes) was calculated, and thereafter, 2,577 probes having a standard deviation in the β value distribution that was greater than 0.25 were used to carry out an unsupervised hierarchical cluster analysis (FIG. 3).

As a result of the above described analysis, the analysis target cases were classified into two groups, namely, an HMCC group (17 cases) having a high methylation level and an LMCC group (35 cases) having a low methylation level.

The response rate to a high EGFR antibody drug was compared between the above described two groups (HMCC group and LMCC group) (Table 2). When the response rate to the anti-EGFR antibody drug has been focused, the response rate of the LMCC group was found to be 34% (12 cases), whereas the response rate of the HMCC group was found to be 6% (1 case). Thus, the response rate of the LMCC group was significantly high (p=0.03).

TABLE 2
Comparison of response rate to anti-EGFR antibody
drug in Cohort 2 between two groups

| | All samples | | HMCC group | | LMCC group | | |
|---|---|---|---|---|---|---|---|
| | Number of | | Number of | | Number of | | p |
| | subjects | % | subjects | % | subjects | % | value |
| RR (%) | | 25.0 | | 5.9 | | 34.3 | 0.03 |
| CR | 0 | 0 | 0 | 0 | 0 | 0 | |
| PR | 13 | 25.0 | 1 | 5.9 | 12 | 34.3 | |
| SD | 22 | 42.3 | 7 | 41.2 | 15 | 42.9 | |
| PD | 17 | 32.7 | 9 | 52.9 | 8 | 22.9 | |
| NA | 0 | | 0 | | 0 | | |

CR: complete response,
PR: partial response,
SD: stable,
PD: progressed,
RR: response rate

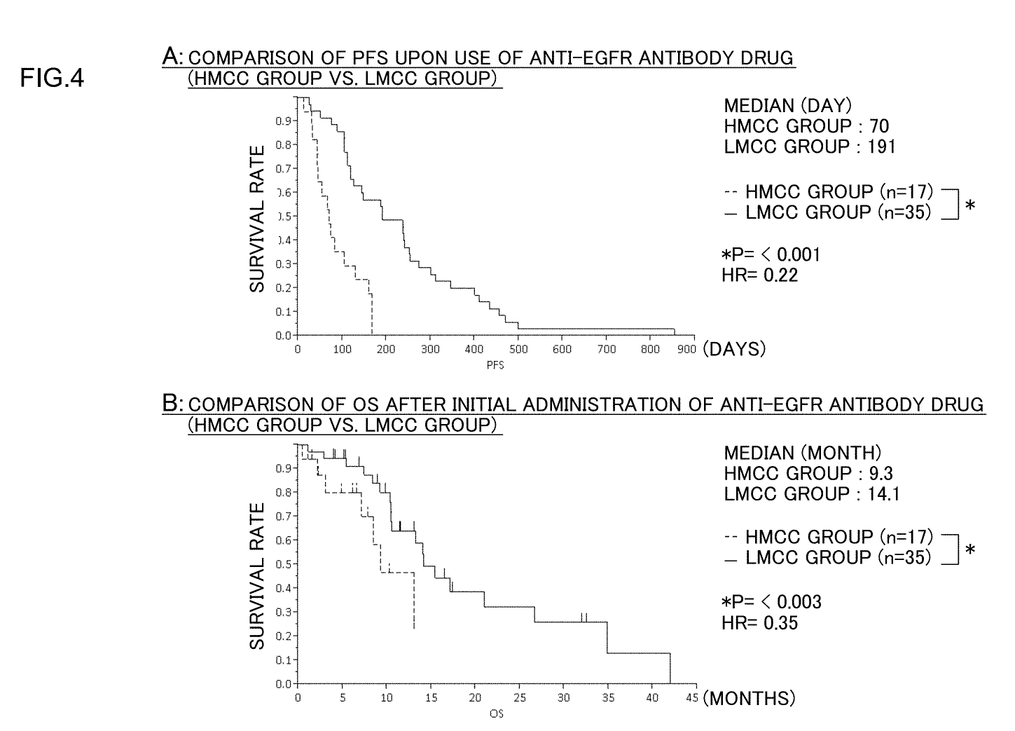

When the progression-free survival (PFS) upon the use of an anti-EGFR antibody drug was focused, the median of the LMCC group was found to be 191 days, whereas the median of the HMCC group was found to be 70 days. Thus, the progression-free survival (PFS) was significantly prolonged in the LMCC group (p=<0.001, HR=0.22: FIG. 4A).

With regard to a comparison in terms of the overall survival (OS) after completion of the initial administration of an anti-EGFR antibody drug, the median of the LMCC group was found to be 14.1 months, whereas the median of the HMCC group was found to be 9.3 months. Thus, the overall survival (OS) was significantly prolonged in the LMCC group (p=0.03, HR=0.35: FIG. 4B).

From the aforementioned results, a significant difference was found in all of response rate, PFS, and OS, upon the use of an anti-EGFR antibody drug, between the two groups that had been classified based on the methylation state, and thus, the role of the comprehensive methylation state demonstrated in Example 1 as a factor for predicting the therapeutic effects of an anti-EGFR antibody drug was reproduced.

### Example 3: Comparison with Existing Biomarkers

As described above, in recent years, it has been reported that an anti-EGFR antibody drug provides insufficient therapeutic effects on a case having mutations on KRAS exons 2, 3 and 4, NRAS exons 2, 3 and 4, as well as KRAS exon 2. Thus, the present antibody has been clinically applied as a biomarker in Japan these days.

Out of 97 analysis target cases in the present study, 49 cases were also subjected to whole exon analysis. Thus, in terms of prediction of the therapeutic effects of an anti-EGFR antibody drug, a comparison was made between the present classification based on methylation and classification using existing biomarkers (the aforementioned KRAS and NRAS are collectively referred to as a RAS genotype) (Table 3).

TABLE 3
Comparison of response rate to anti-EGFR antibody drug between
two groups based on present classification and RAS genotype

| | Present classification | | | | |
|---|---|---|---|---|---|
| | HMCC group (n = 13) | | LMCC group (n = 36) | | |
| | Number of | | Number of | | |
| | subjects | % | subjects | % | p value |
| RR (%) | | 7.7 | | 33.3 | 0.07 |
| CR | 0 | 0 | 0 | 0 | |
| PR | 1 | 7.7 | 12 | 33.3 | |
| SD | 7 | 53.8 | 16 | 44.4 | |
| PD | 5 | 38.5 | 8 | 22.2 | |
| NA | 0 | 0 | 0 | 0 | |

| | RAS genotype | | | | |
|---|---|---|---|---|---|
| | mutant RAS group | | wild-type RAS group | | |
| | (n = 13) | | (n = 36) | | |
| | Number of | | Number of | | |
| | subjects | % | subjects | % | p value |
| RR (%) | | 7.7 | | 33.3 | 0.07 |
| CR | 0 | 0 | 0 | 0 | |
| PR | 1 | 7.7 | 12 | 33.3 | |
| SD | 5 | 38.5 | 18 | 50.0 | |
| PD | 7 | 53.8 | 6 | 16.7 | |
| NA | 0 | | 0 | 0 | |

CR: complete response,
PR: partial response,
SD: stable,
PD: progressed,
RR: response rate

First, a comparison was made in terms of the response rate to the anti-EGFR antibody drug. Treatment-resistant groups, namely, an HMCC group and a mutant RAS group both had a response rate of 7.7%, and on the other hand, treatment-sensitive groups, namely, an LMCC group and a wild-type RAS group both had a response rate of 33.3%. From these results, it was demonstrated that the present classification exhibits a correlation with the response rate to the anti-EGFR antibody drug, at a level equivalent to the classification based on the RAS genotype.

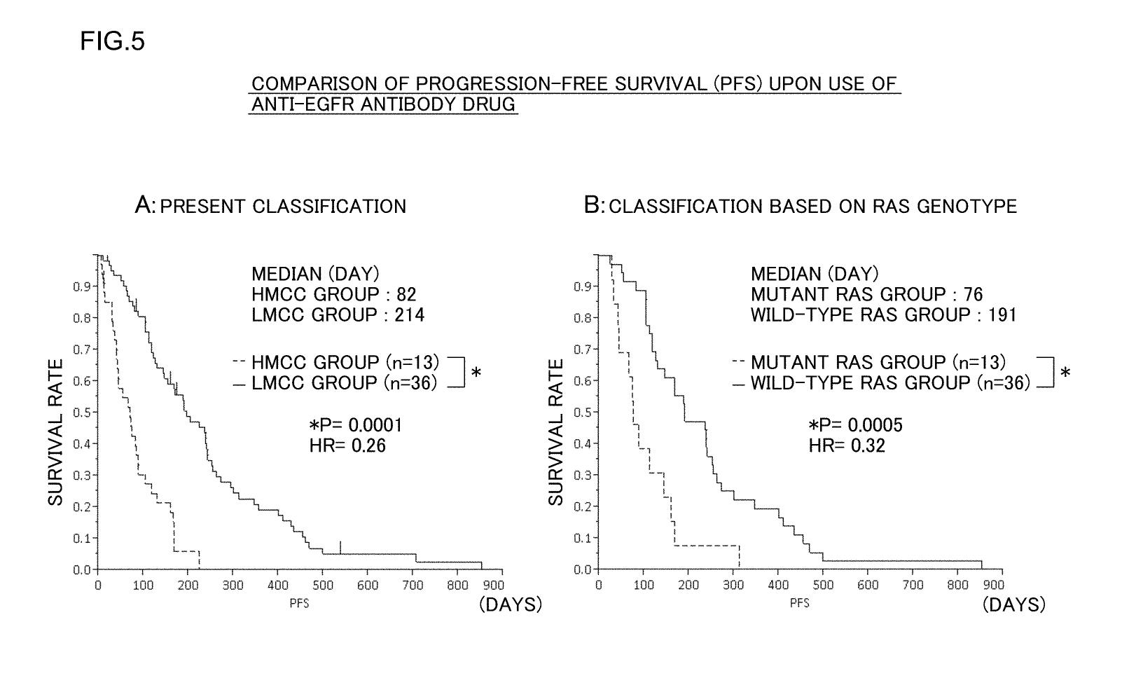

Subsequently, in terms of the progression-free survival (PFS) upon the use of an anti-EGFR antibody drug, a comparison was made (FIG. 5). In both of the classifications, PFS was significantly prolonged in the treatment-sensitive groups (LMCC group and wild-type RAS group). The hazard ratios (HR) were found to be 0.26 (LMCC group vs. HMCC group) and 0.32 (wild-type RAS group vs. mutant RAS group), respectively. From these results, it was demonstrated that the present classification exhibits a correlation with PFS upon the use of the anti-EGFR antibody drug, at a level equivalent to the classification based on the RAS genotype.

A multivariate analysis was carried out using factors possibly having an influence on the progression-free survival (PFS) upon the use of an anti-EGFR antibody drug (Table 4). The hazard ratio (HR) of the present classification based on the methylation state, in which the p value was lower than 0.05, was equivalent to the hazard ratio (HR) of the classification based on RAS genotype, in which the p value was lower than 0.05. From these results, it was demonstrated that the present classification is an independent determinant for the PFS upon the use of the anti-EGFR antibody drug, and also that the hazard ratio of the present classification is equivalent to that of the classification based on the RAS genotype.

TABLE 4
Multivariate analysis of using Cox proportional hazard
models on PFS upon use of anti-EGFR antibody drug

| | HR | P-value |
|---|---|---|
| Methylation state (HMCC group vs. LMCC group) | 0.36 | 0.03 |
| RAS genotype (mutant group vs. wild-type group) | 0.36 | 0.02 |
| Age (less than 61 years old vs. 61 years old or more) | 0.81 | 0.55 |
| Sex (male vs. female) | 0.94 | 0.88 |
| Primary site (distal colon vs. proximal colon) | 1.36 | 0.46 |
| Clinical stage upon excision of primary lesion (stage III | 0.75 | 0.40 |
| or lower vs. stage IV) | | |
| Number or organs having metastasis (1 or less vs. 2 or | 1.36 | 0.37 |
| more) | | |
| Number of previous regimens (2 or less vs. 3 or more) | 1.25 | 0.72 |
| BRAF mutation (without vs. with) | 0.17 | 0.07 |

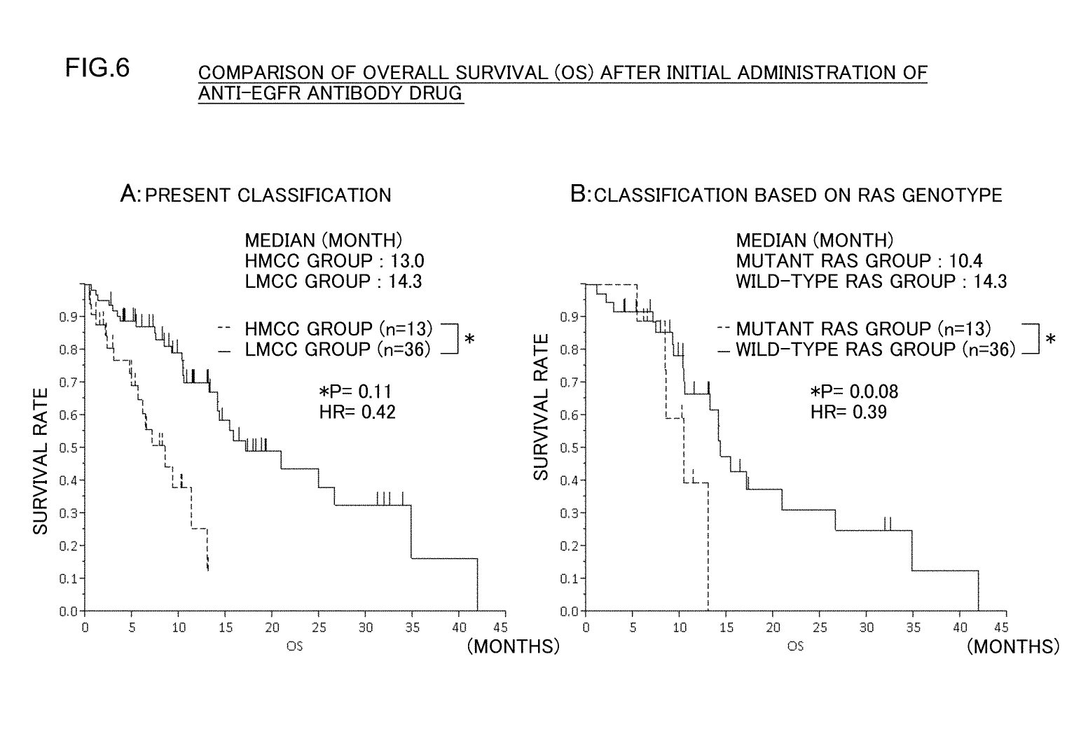

In terms of the overall survival (OS) after completion of the initial administration of an anti-EGFR antibody drug, a comparison was made (FIG. 6). In both of the two classifications, OS tended to be prolonged in treatment-sensitive groups (LMCC group and wild-type RAS group). The hazard ratios (HR) were found to be 0.42 (LMCC group vs. HMCC group) and 0.39 (wild-type RAS group vs. mutant RAS group). In both of the classification methods, a significant difference was not found between the two groups. However, the present classification exhibited a correlation even with OS after completion of the initial administration of the anti-EGFR antibody drug, at a level equivalent to the classification based on the RAS genotype.

As described above, the present classification exhibited a correlation with all of the response rate to an anti-EGFR antibody drug, PFS upon the use of an anti-EGFR antibody drug, and OS after completion of the initial administration of an anti-EGFR antibody drug, at levels equivalent to the classification based on the RAS genotype. Moreover, from the results of the multivariate analysis, it was found that the present classification is a determinant independent from the RAS genotype, with regard to PFS upon the use of an anti-EGFR antibody drug.

### Example 4: Comparison with Known Subtype Classification

Yagi et al. have classified colorectal cancer into three subtypes ((HME (highly-methylated group), IME (intermediately-methylated group), and LME (low-methylated group)) by examining the methylation states of 7 genes, and have then demonstrated that cases having a KRAS mutation are concentrated in IME (cited above: Yagi K. et al., Clin Cancer Res. 2010 Jan. 1; 16(1): 21-33). Moreover, Yagi et al. have also demonstrated that the overall survival is significantly reduced in cases having IME and a KRAS mutation, in compared with other case groups.

These 7 genes were evaluated in terms of methylation states in our case groups, and the genes were then classified into the three groups in accordance with the method described in the aforementioned study paper.

From 6 out of the 7 genes used in the subtype classification, probes contained in the region analyzed by Yagi et al. were extracted. However, in the case of the remaining one gene (FBN2), since probes contained in the region evaluated by Yagi et al. had not been designed, probes that were located close to the region evaluated by Yagi et al. were extracted from probes contained in the same CpG island, using the browser of UCSC.

A plurality of probes were extracted from each marker. Thus, when there were, for example, 3 probes, if a majority (two or more) of probes was found to be methylated (β value≧0.5), the marker was considered positive to methylation.

As a result, a total of 97 cases from Example 1 and Example 2 were classified into 3 groups, namely, into HME (7 cases), IME (16 cases) and LME (74 cases) (Table 5).

TABLE 5
Comparison of response rate to anti-
EGFR antibody between two groups

| | Present classification | | | | |
|---|---|---|---|---|---|
| | HMCC | | LMCC | | |
| | Number of | | Number of | | |
| | subjects | % | subjects | % | p value |
| RR (%) | | 6.1 | | 34.9 | 0.002 |
| CR | 0 | 0 | 0 | 0 | |
| PR | 2 | 6.1 | 22 | 34.9 | |
| SD | 11 | 33.3 | 26 | 41.3 | |
| PD | 20 | 60.6 | 15 | 23.8 | |
| NA | 1 | | 0 | | |
| | Yagi classification | | | | |
| | HME + IME | | LME | | |
| | Number of | | Number of | | |
| | subjects | % | subjects | % | p value |
| RR (%) | | 4.5 | | 31.1 | 0.01 |
| CR | 0 | 0 | 0 | 0 | |
| PR | 1 | 4.5 | 23 | 31.1 | |
| SD | 7 | 31.8 | 30 | 40.5 | |
| PD | 14 | 63.6 | 21 | 28.4 | |
| NA | 1 | | 0 | 0 | |

CR: complete response,
PR: partial response,
SD: stable,
PD: progressed,
RR: response rate

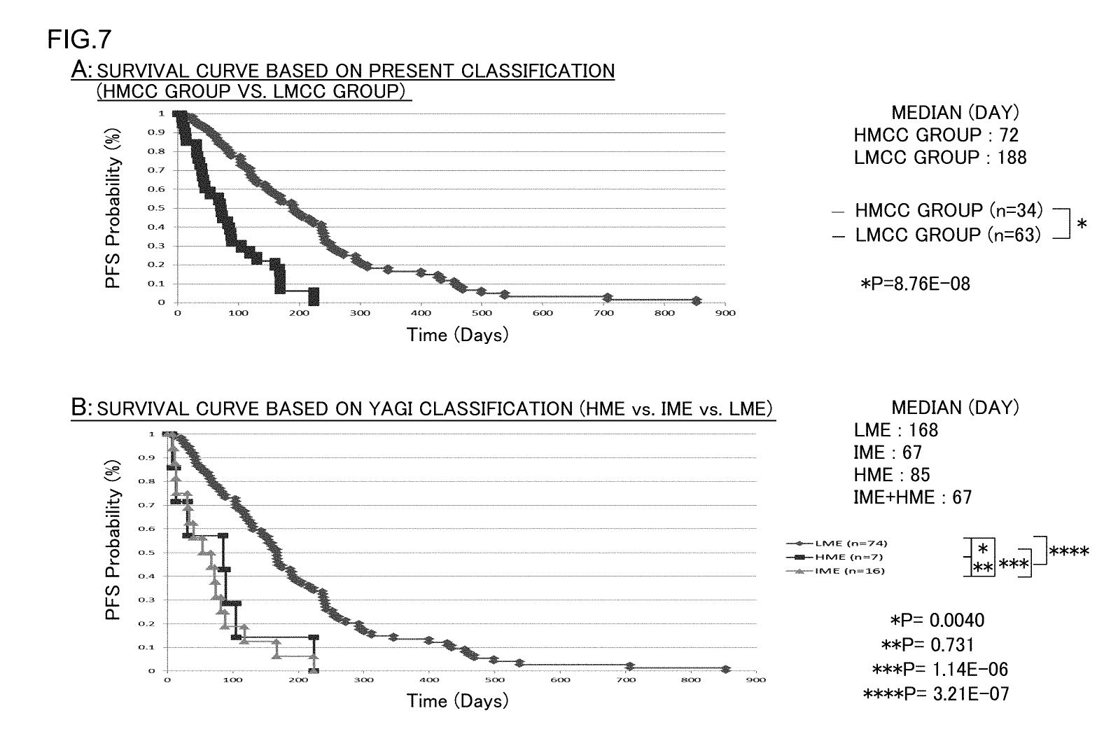

The median of the progression-free survival (PFS) upon the use of an anti-EGFR antibody drug was 85 days in HME, 67 days in IME, and 168 days in LME. Thus, the results demonstrated that the progression-free survival (PFS) was significantly prolonged in LME, in comparison to the two other groups HME and IME (vs. HME, p=0.004, vs. IME, p=1.14E-06, vs. HME+IME, p=3.21E-07: FIG. 7B).

From the aforementioned results, it was demonstrated that it is sufficiently possible to predict the therapeutic effects of an anti-EGFR antibody drug based on methylation profiles, even by narrowing the number of used probes to several probes, and it was also demonstrated that the conventional diagnostic method based on comprehensive analysis can be converted to a simple diagnostic method involving detection of methylation in a limited region, which is ready for practical use.

In addition, 23 cases included in HME and IME were all included in the highly-methylated group in Examples 1 and 2.

The present Example demonstrated that the classification method of the present invention can extract many methylated cases, in comparison to the existing subtype classification based on methylation, and that even some highly-methylated cases, which could not be extracted by the existing subtype classification, are shown to be resistant to the anti-EGFR antibody drug. That is to say, according to the method of the present invention, therapeutic sensitivity to the anti-EGFR antibody drug can be predicted with higher precision than that of the existing subtype classification.

There are a total of 34 highly-methylated group cases that are drug resistant groups in Examples 1 and 2. It was considered that 11 cases, which may be drug resistant cases included in LME, can be extracted by adding some more markers to the markers associated with the 7 genes used in Example 3.

### Example 5: Studies Regarding Classification Method Using a Limited Number of Probes

Using 97 cases included in Example 1 and Example 2, a classification method using a limited number of probes was studied. In Examples 1 and 2, the extracted 3,163 and 2,577 probes were used in each analysis, and the target cases were classified according to an unsupervised cluster analysis. Among the probes used in the analysis in each Example, 1,744 probes were common in Examples 1 and 2. From these 1,744 probes, 1,053 probes having a difference in the β value between the case group classified into the HMCC group and the case group classified into the LMCC group were extracted (Table 7: shown at the end of the Examples).

From the thus extracted 1,053 probes, 4 to 10 probes were randomly extracted, and the cases were then classified into an HMCC group or an LMCC group, depending on the methylation states of the extracted probes. With regard to determination of the methylation of each probe, when the probe had a β value of 0.5 or greater, it was determined that the probe was positive to methylation, and when the probe had a β value of less than 0.5, it was determined that the probe was negative to methylation.

When 60% or more of the probes used in the analysis were methylation-positive, the case was classified into an HMCC group (for example, the case is classified into an HMCC group, if 3 or more of the used 4 probes are methylation-positive, or 4 or more of the used 6 probes are methylation-positive).

Regarding the results classified by the above described method, the classification results of each case in Examples 1 and 2 were assumed to be correct, and sensitivity and specificity were calculated. Specifically, the sensitivity indicates the ratio of the cases considered to be among an HMCC group also by the method of the present Example to a total of 34 cases considered to be among an HMCC group in Examples 1 and 2. On the other hand, the specificity indicates the ratio of the cases considered to be among an LMCC group also by the method of Example 5 to a total of 63 cases considered to be among an LMCC group in Examples 1 and 2.

As the number of probes extracted, five numbers were set (4, 5, 6, 7, and 10). Extraction of any given probes, classification of cases, and calculation of sensitivity and specificity were defined as 1 set, and 5 sets of these operations were repeatedly carried out under individual conditions, and the mean value thereof was then defined as sensitivity or specificity under individual conditions. The sensitivity and specificity calculated under individual conditions are shown in the following table.

TABLE 6

| | 4_3 | 5_3 | 6_4 | 7_5 | 10_6 |
|---|---|---|---|---|---|
| Sensitivity | 83.54% | 90% | 87.06% | 83.52% | 90.12% |
| Specificity | 93.66% | 90.80% | 91.78% | 97.12% | 95.26% |

The numbers shown in the form of X_Y in the uppermost row of each column indicate determination conditions. That is, the numbers X_Y indicate that, among the randomly extracted X probes, a Y number or more of probes are methylation-positive (e.g.: “4_3” indicates that 3 or more of the extracted 4 probes are methylation-positive).

From these results, it was demonstrated that a case group can be classified with sensitivity of 83.5% and specificity of 93.7%, by randomly extracting at least 4 probes from a list of the extracted 1,053 probes.

From the above described results, it was demonstrated that the therapeutic effects of an anti-EGFR antibody drug can be more simply predicted with sensitivity and specificity sufficiently suitable for practical use, by evaluating the methylation states of several probes selected from the list of 1,053 probes shown in Table 7.

### Example 6: Correlation of Treatment Results with Methylation Classification in Metastatic Colorectal Cancer

1) Correlation of the Results of Primary Treatment with Methylation Classification

A comprehensive methylation analysis was carried out on 94 metastatic colorectal cancer cases according to Example 1, and the cases were classified into an HMCC group (34 cases) and an LMCC group (60 cases). The two groups were compared with each other in terms of progression-free survival after a primary treatment.

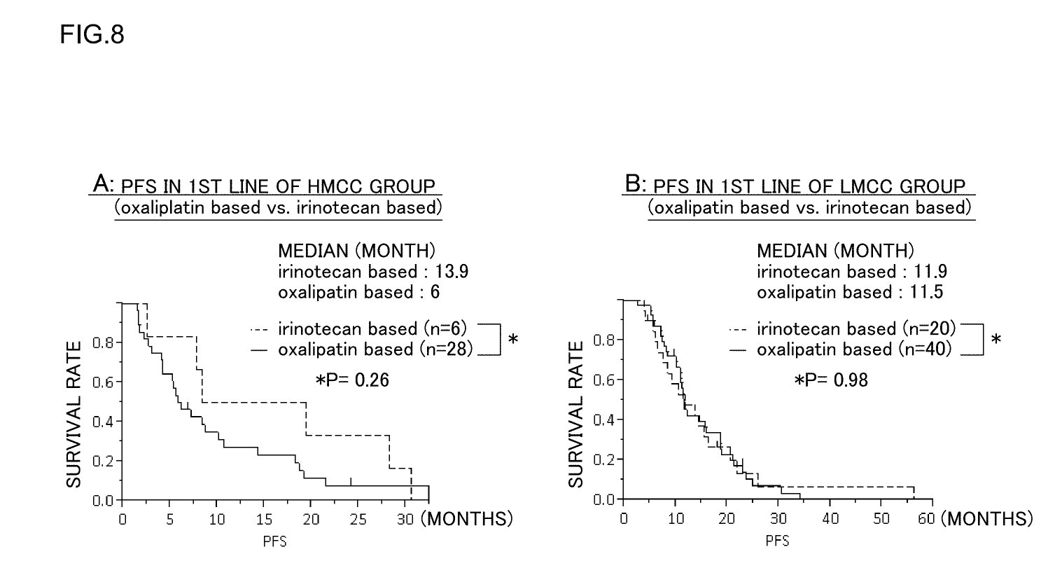

As a result, in the HMCC group, the progression-free survival tended to be shorter in the case of a combination therapy comprising oxaliplatin (solid line) than in the case of a combination therapy comprising irinotecan (broken line), and on the other hand, in the LMCC group, such a difference in the progression-free survival was not found between the two therapies (FIG. 8). Accordingly, the methylation classification of the present invention was considered to be useful as a biomarker for selection of treatment in the primary treatment for metastatic colorectal cancer.

2) Correlation of the Results of Secondary Treatment with Methylation Classification

A comprehensive methylation analysis was carried out on 84 metastatic colorectal cancer cases, and the cases were classified into an HMCC group (31 cases) and an LMCC group (53 cases). Then, the two groups were compared with each other in terms of progression-free survival after a secondary treatment.

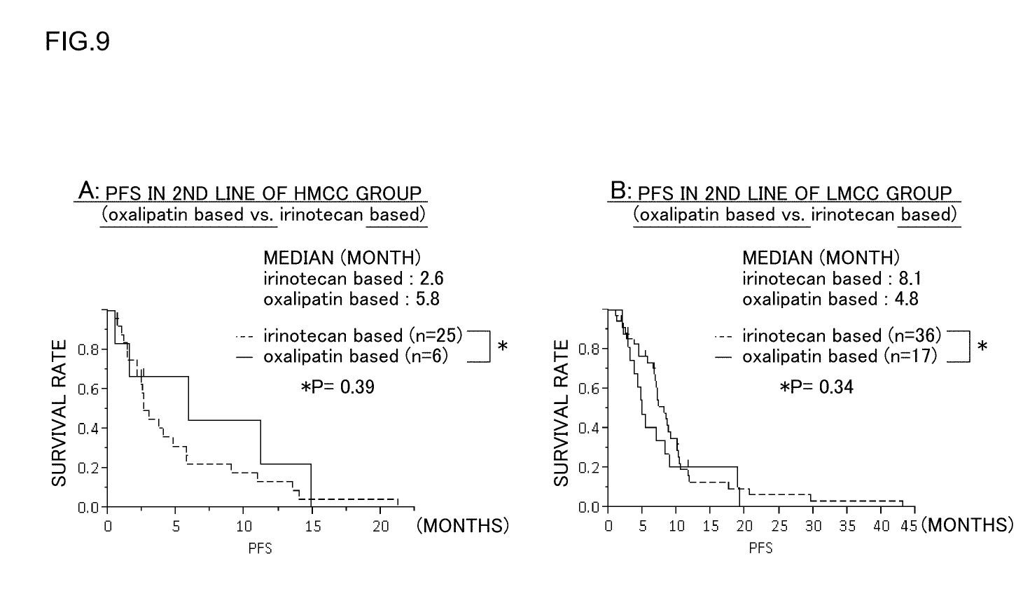

As a result, in the HMCC group, the progression-free survival tended to be shorter in the case of a combination therapy comprising irinotecan (broken line) than in the case of a combination therapy comprising oxaliplatin (solid line), and on the other hand, in the LMCC group, the progression-free survival tended to be shorter in the case of a combination therapy comprising oxaliplatin (solid line) than in the case of a combination therapy comprising irinotecan (broken line) (FIG. 9). From the aforementioned results, the methylation classification of the present invention was considered to be useful as a biomarker for selection of treatment in the secondary treatment for metastatic colorectal cancer.

3) Correlation of the Results of the Primary and Secondary Therapies with Methylation Classification

A comprehensive methylation analysis was carried out on 84 metastatic colorectal cancer cases, and the cases were classified into an HMCC group (31 cases) and an LMCC group (53 cases). Then, the two groups were compared with each other in terms of the treatment results of a combination therapy comprising oxaliplatin or irinotecan in the primary and secondary therapies, and the overall survival.

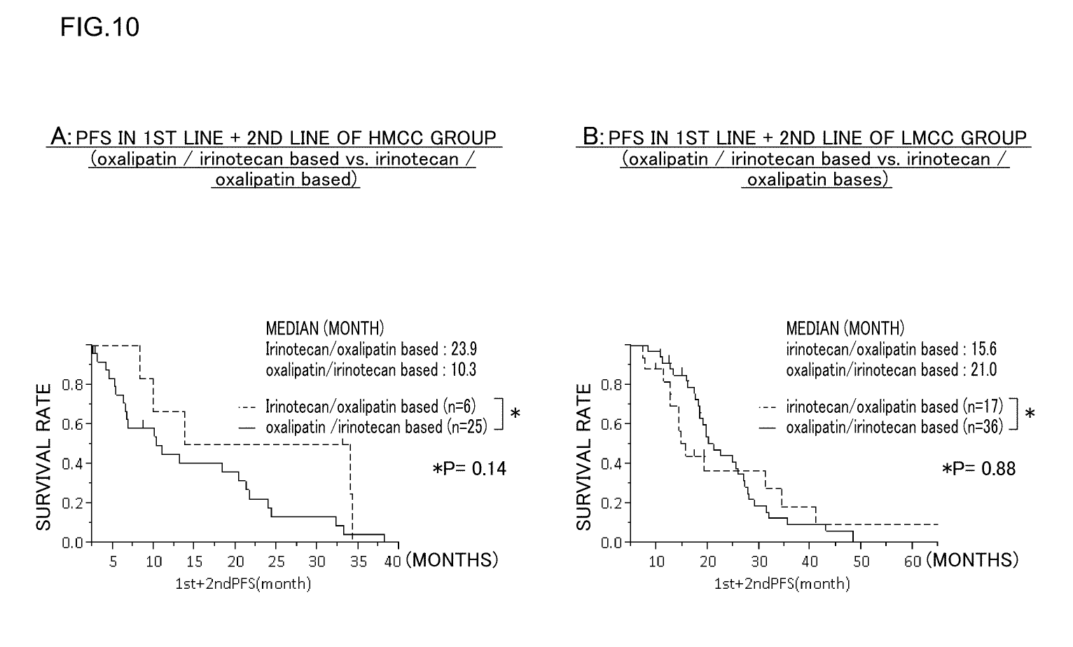

As a result, in the HMCC group, the progression-free survival tended to be shorter in a group (solid line) on which a combination therapy comprising oxaliplatin was carried out as a primary treatment and also comprising irinotecan was then carried out as a secondary treatment, than in a group (broken line) on which the therapies were carried out in the opposite order (FIG. 10A). On the other hand, in the LMCC group, a difference in the progression-free survival was not found between the two therapies (FIG. 10B).

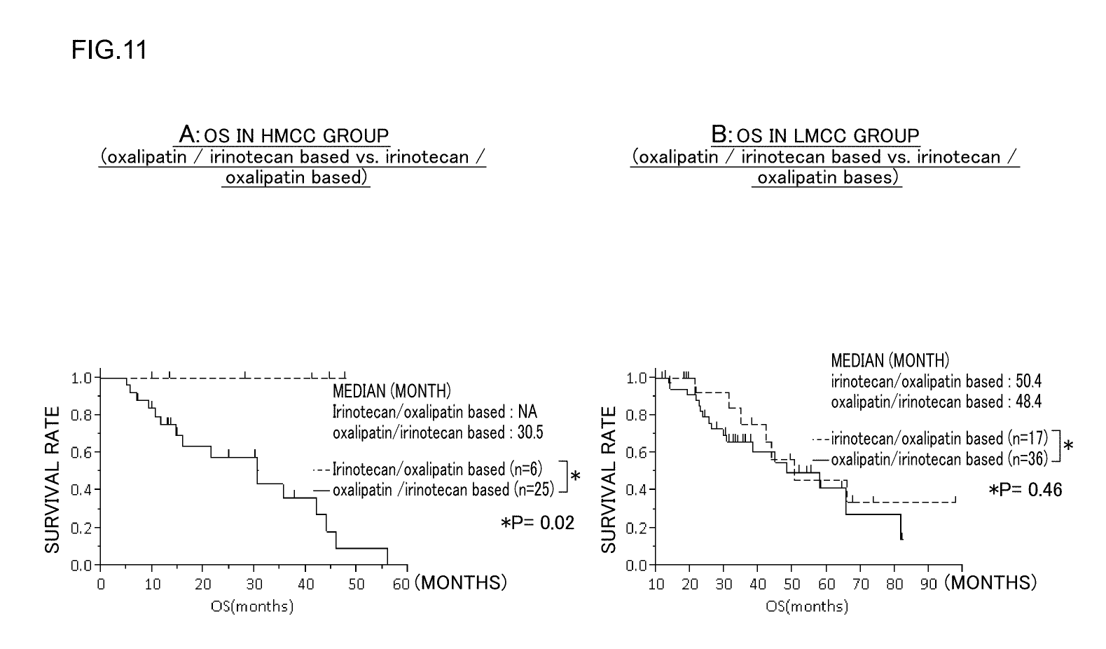

Moreover, in the HMCC group, the overall survival was significantly shorter in a group (solid line) on which a combination therapy comprising oxaliplatin was carried out as a primary treatment and also comprising irinotecan was then carried out as a secondary treatment, than in a group (broken line) on which the therapies were carried out in the opposite order (FIG. 11A). On the other hand, in the LMCC group, a difference in the overall survival was not found between the two therapies (FIG. 11B).

As described above, the present methylation classification was considered to be useful as a biomarker, not only for selection of treatment in the primary treatment and the secondary treatment for metastatic colorectal cancer, but also for selecting the order in which the primary treatment and the secondary treatment were applied.

### Example 7: Correlation of Treatment Results with CIMP Classification in Metastatic Colorectal Cancer

1) Correlation of the Results of Primary Treatment with CIMP Classification

A CIMP analysis was carried out on 108 metastatic colorectal cancer cases according to a known method, and the cases were classified into a CIMP-positive group (24 cases) and a CIMP-negative group (84 cases). Then, the two groups were compared with each other in terms of progression-free survival after a primary treatment.

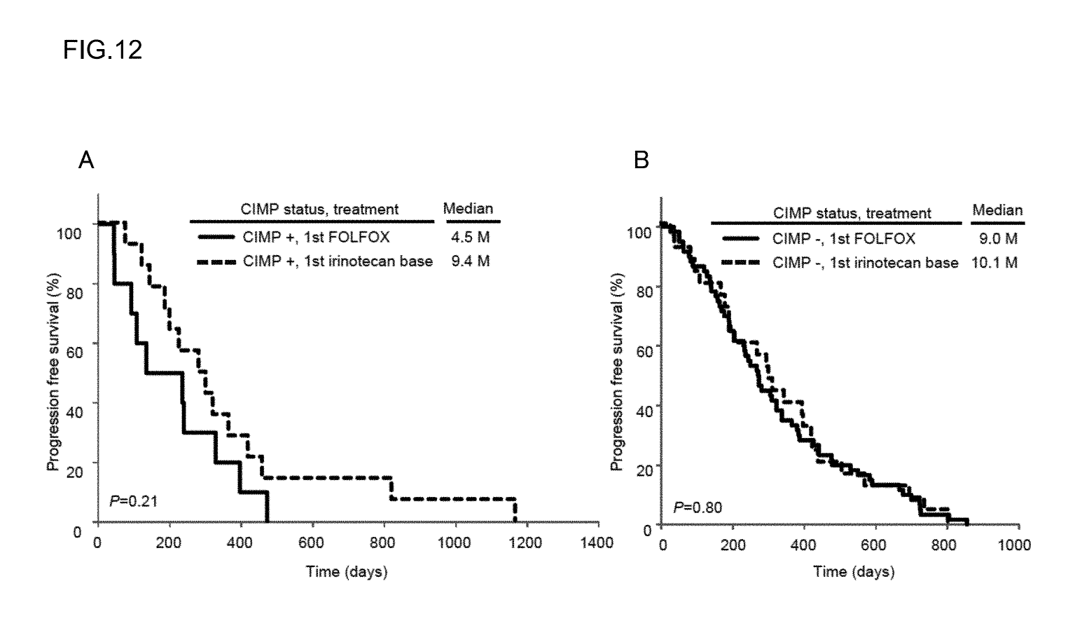

In the CIMP-positive group, the progression-free survival tended to be shorter in the case of a combination therapy comprising oxaliplatin (solid line) than in the case of a combination therapy comprising irinotecan (broken line). On the other hand, in the CIMP-negative group, a difference in the progression-free survival was not found between the two therapies (FIG. 12). Accordingly, the CIMP classification was considered to be useful as a biomarker for selection of treatment in the primary treatment for metastatic colorectal cancer.

2) Correlation of the Results of Secondary Treatment with CIMP Classification

A CIMP analysis was carried out on 78 metastatic colorectal cancer cases, and the cases were classified into a CIMP-positive group (17 cases) and a CIMP-negative group (61 cases). Then, the two groups were compared with each other in terms of progression-free survival after a secondary treatment.

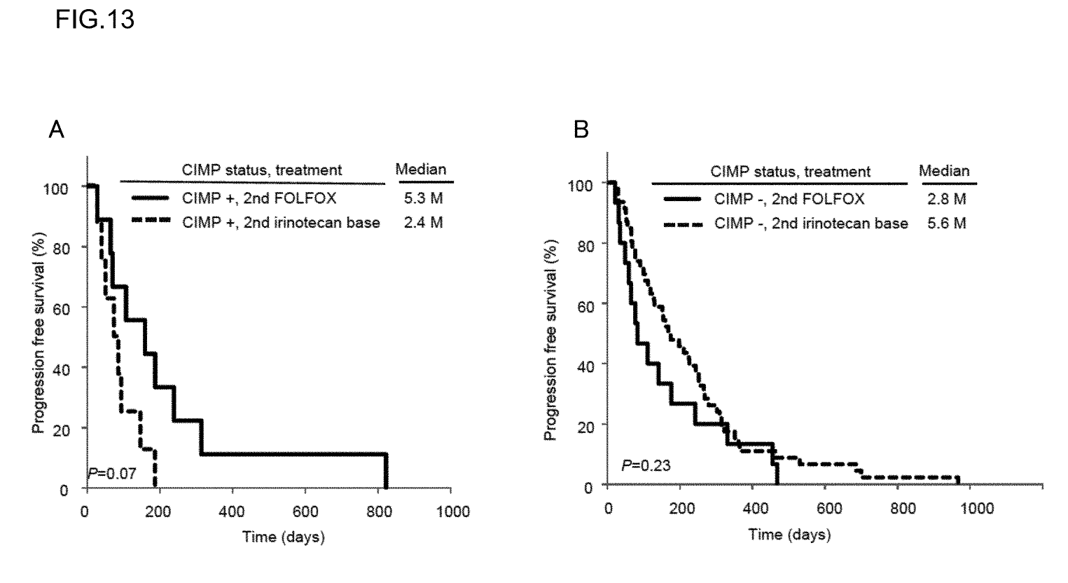

As a result, in the CIMP-positive group, the progression-free survival tended to be shorter in the case of a combination therapy comprising irinotecan (solid line) than in the case of a combination therapy comprising oxaliplatin (broken line) (FIG. 13A). On the other hand, in the CIMP-negative group, a difference in the progression-free survival was not found between the two therapies (FIG. 13B). Accordingly, the CIMP classification was considered to be useful as a biomarker for selection of treatment in the secondary treatment for metastatic colorectal cancer.

3) Correlation of the Results of Primary and Secondary Therapies with CIMP Classification

A CIMP analysis was carried out on metastatic colorectal cancer (78 cases), and the cases were classified into a CIMP-positive group (17 cases) and a CIMP-negative group (61 cases). Then, the two groups were compared with each other in terms of the treatment results of a combination therapy comprising oxaliplatin or irinotecan in the primary and secondary therapies.

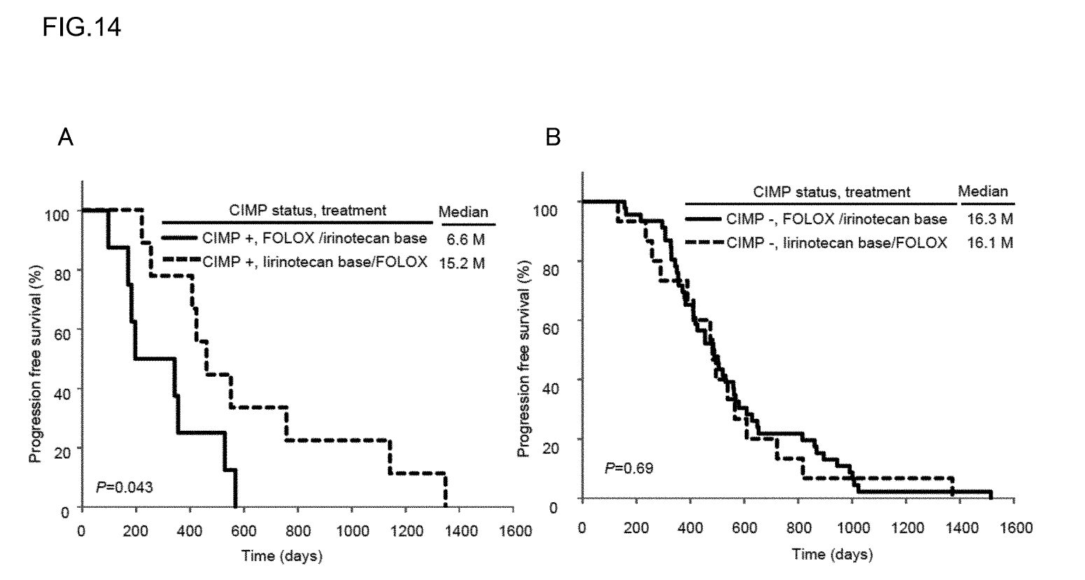

As a result, in the CIMP-positive group, the progression-free survival was significantly shorter in a group (solid line) on which a combination therapy comprising oxaliplatin was carried out as a primary treatment and also comprising irinotecan was then carried out as a secondary treatment, than in a group (broken line) on which the therapies were carried out in the opposite order (FIG. 14A). On the other hand, in the CIMP-negative group, a difference in the progression-free survival was not found between the two therapies (FIG. 14B).

A CIMP analysis was carried out on 108 metastatic colorectal cancer cases on which a primary treatment had been carried out, and also on 78 metastatic colorectal cancer cases which had been subjected to a secondary treatment. Thus, the 108 cases were classified into a CIMP-positive group (24 cases) and a CIMP-negative group (84 cases), and the 78 cases were classified into a CIMP-positive group (17 cases) and a CIMP-negative group (61 cases).

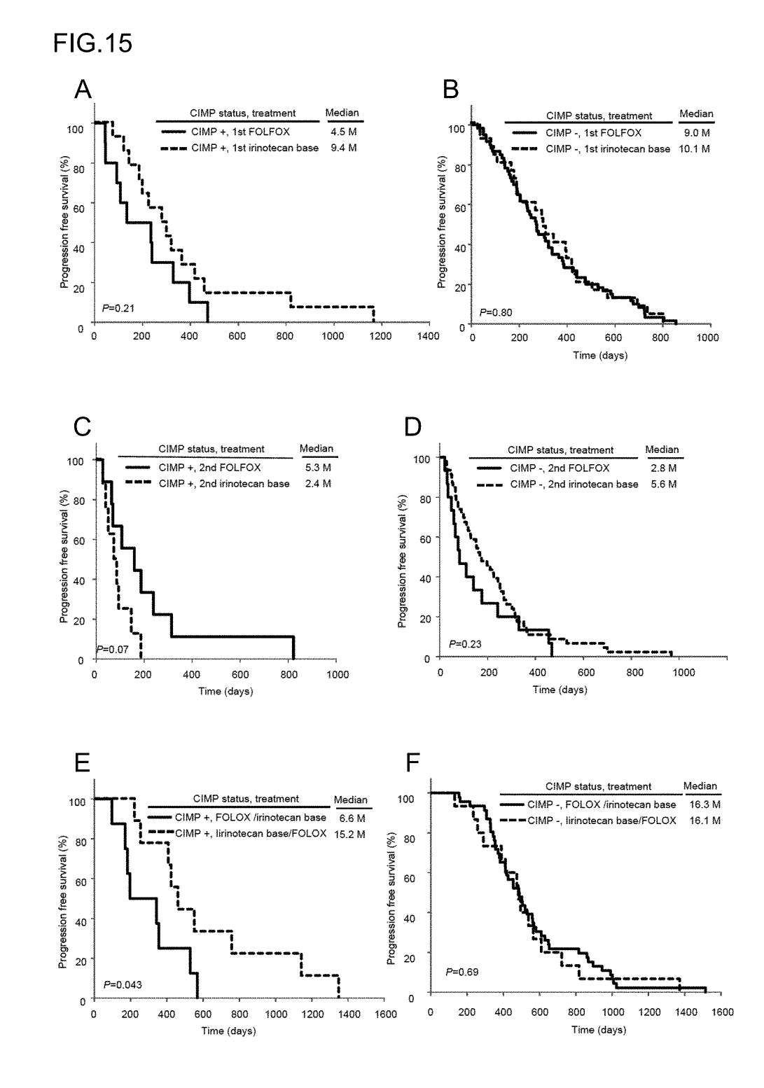

In the CIMP-positive cases, the progression-free survival tended to be short in a group on which a combination therapy comprising oxaliplatin was carried out as a primary treatment and also comprising irinotecan was then carried out as a secondary treatment (FIGS. 15A and C). Meanwhile, when the analysis was continuously carried out from the primary treatment to the secondary treatment, it was found that the progression-free survival was significantly shorter in a group on which a combination therapy comprising oxaliplatin was carried out as a primary treatment and also comprising irinotecan was then carried out as the subsequent secondary treatment, than in a group on which the therapies were carried out in the opposite order (FIG. 15E). In the CIMP-negative group, a difference in the progression-free survival was not found between the two therapies (FIGS. 15B, D and F).

As described above, the CIMP classification was also considered to be useful as a biomarker, not only for selection of treatment in the primary treatment and the secondary treatment for metastatic colorectal cancer, but also for selecting the order in which the primary treatment and the secondary treatment were applied.

TABLE 7

| TargetID | CHR | MAPINFO | UCSC_REFGENE_NAME |
|---|---|---|---|
| cg00024472 | 17 | 47573864 | NGFR |
| cg00181968 | 1 | 179712204 | FAM163A |
| cg00263760 | 10 | 118897366 | VAX1; VAX1 |
| cg00268840 | 11 | 44332653 | ALX4 |
| cg00278028 | 22 | 28196834 | MN1; MN1 |
| cg00281842 | 5 | 140855562 | PCDHGA4; PCDHGA12; PCDHGA11; PCDHGA11; PCDHGA9; |
| | | | PCDHGA1; PCDHGB1; PCDHGC3; PCDHGB6; PCDHGB3; |
| | | | PCDHGB7; PCDHGC3; PCDHGA6; PCDHGA8; PCDHGA10; |
| | | | PCDHGA5; PCDHGB4; PCDHGA3; PCDHGC3; PCDHGA2; |
| | | | PCDHGA7; PCDHGB2; PCDHGB5 |
| cg00290506 | 1 | 224804226 | CNIH3; CNIH3 |
| cg00295794 | 13 | 100641409 | |
| cg00366818 | 10 | 28287879 | ARMC4 |
| cg00393798 | 11 | 8615694 | STK33 |
| cg00405843 | 7 | 128828575 | SMO |
| cg00433770 | 19 | 17392656 | ANKLE1; ANKLE1 |
| cg00450824 | 11 | 8615685 | STK33 |
| cg00462168 | 15 | 79724794 | KIAA1024 |
| cg00498155 | 19 | 37157879 | ZNF461 |
| cg00500564 | 10 | 128994030 | FAM196A; DOCK1 |
| cg00513060 | 19 | 58111480 | ZKF530 |
| cg00544449 | 15 | 79724802 | KIAA1024 |
| cg00563873 | 8 | 93115461 | |
| cg00592781 | 12 | 66122874 | |
| cg00652796 | 3 | 96532832 | EPHA6 |
| cg00660608 | 10 | 125853219 | |
| cg00662647 | 1 | 234350051 | SLC35F3 |
| cg00714184 | 4 | 155664242 | LRAT |
| cg00755058 | 13 | 96296844 | DZIP1; DZIP1; DZIP1; DZIP1 |
| cg00765312 | 12 | 85674694 | ALX1 |
| cg00790098 | 2 | 223167400 | CCDC140 |
| cg00810956 | 3 | 27771766 | |
| cg00866976 | 16 | 58224782 | GNAO1; GNAO1; LOC283856 |
| cg00880018 | 13 | 43149171 | TNFSF11; TNFSF11 |
| cg00881552 | 14 | 90527990 | KCNK13 |
| cg00912625 | 3 | 2140977 | CNTN4 |
| cg00922781 | 10 | 128077080 | ADAM12; ADAM12; ADAM12; ADAM12 |
| cg00945293 | 1 | 2984869 | FLJ42875; PRDM16; PRDM16; FLJ42875 |
| cg00962913 | 1 | 179712281 | FAM163A |
| cg00973653 | 1 | 70033627 | |
| cg00981837 | 5 | 33936262 | RXFP3 |
| cg00995327 | 3 | 142838847 | CHST2; CHST2 |
| cg01036409 | 17 | 66597372 | FAM20A |
| cg01043019 | 6 | 127440510 | RSPO3 |
| cg01051310 | 1 | 228194443 | WNT3A |
| cg01084239 | 11 | 8102510 | TUB; TUB |
| cg01086895 | 11 | 6676515 | DCHS1 |
| cg01088410 | 5 | 170739179 | |
| cg01163842 | 14 | 95235125 | GSC |
| cg01188592 | 20 | 982831 | RSPO4; RSPO4; RSPO4; RSPO4 |
| cg01193217 | 8 | 145104575 | |
| cg01229798 | 10 | 88126853 | GRID1 |
| cg01261798 | 10 | 134000034 | DPYSL4 |
| cg01277542 | 16 | 55689901 | SLC6A2 |
| cg01295203 | 8 | 70984199 | PRDM14 |
| cg01366595 | 5 | 42952307 | |
| cg01379240 | 3 | 138666144 | C3orf72; FOXL2; C3orf72 |
| cg01454215 | 2 | 27530363 | UCN |
| cg01545587 | 14 | 105993579 | TMEM121 |
| cg01555431 | 6 | 151562026 | AKAP12 |
| cg01559617 | 1 | 107684059 | NTNG1; NTNG1; NTNG1 |
| cg01616178 | 8 | 41755140 | ANK1 |
| cg01649597 | 2 | 232395061 | NMUR1 |
| cg01663018 | 15 | 53097777 | |
| cg01705052 | 16 | 12996234 | SHISA9; SHISA9 |
| cg01718447 | 7 | 30722327 | CRHR2 |
| cg01783070 | 20 | 21686293 | PAX1 |
| cg01791410 | 3 | 150802997 | |
| cg01802453 | 4 | 183370148 | ODZ3 |
| cg01826863 | 2 | 47797953 | KCNK12 |
| cg01886556 | 4 | 17783205 | FAM184B |
| cg01923218 | 11 | 93063888 | CCDC67; CCDC67 |
| cg01950845 | 15 | 93632730 | RGMA; RGMA |
| cg01956420 | 13 | 110959668 | COL4A1; COL4A2; COL4A2 |
| cg01963134 | 12 | 39299364 | CPNE8; CPNE8 |
| cg01969910 | 3 | 179169571 | GNB4 |
| cg01995480 | 22 | 45405827 | PHF21B; PHF21B |
| cg02002231 | 4 | 81187798 | FGF5; FGF5; FGF5; FGF5 |
| cg02009088 | 5 | 139228153 | NRG2; NRG2; NRG2; NRG2 |
| cg02012576 | 12 | 133485691 | |
| cg02040433 | 16 | 58497815 | NDRG4; NDRG4; NDRG4 |
| cg02055132 | 3 | 96533295 | EPHA6 |
| cg02071076 | 4 | 184827086 | STOX2; STOX2 |
| cg02146001 | 20 | 61051548 | GATA5 |
| cg02164129 | 5 | 76926550 | OTP |
| cg02167438 | 20 | 9819697 | PAK7; PAK7 |
| cg02173749 | 2 | 121104029 | INHBB |
| cg02182210 | 20 | 30619096 | C20orf160 |
| cg02229993 | 1 | 166134699 | FAM78B |
| cg02230017 | 16 | 6069019 | A2BP1; A2BP1 |
| cg02246645 | 12 | 103352235 | ASCL1 |
| cg02269161 | 11 | 7273154 | SYT9 |
| cg02282626 | 6 | 30227363 | HLA-L |
| cg02293118 | 7 | 44349704 | CAMK2B; CAMK2B; CAMK2B; CAMK2B; |
| | | | CAMK2B; CAMK2B; CAMK2B; CAMK2B |
| cg02305377 | 12 | 103355958 | |
| cg02325324 | 13 | 23489846 | |
| cg02329935 | 5 | 76506492 | PDE8B; PDE8B; PDE8B; PDE8B; PDE8B |
| cg02330121 | 6 | 118228514 | SLC35F1 |
| cg02367930 | 21 | 32930938 | TIAM1 |
| cg02401454 | 16 | 230343 | HBQ1; HBQ1 |
| cg02403395 | 3 | 192445500 | FGF12 |
| cg02467990 | 7 | 49813102 | VWC2 |
| cg02483484 | 1 | 4716537 | AJAP1; AJAP1 |
| cg02484469 | 20 | 61051036 | GATA5 |
| cg02504416 | 11 | 44331629 | ALX4; ALX4 |
| cg02583418 | 2 | 105473340 | POU3F3 |
| cg02626129 | 13 | 31480646 | C13orf33; C13orf33 |
| cg02678084 | 16 | 4588491 | C16orf5 |
| cg02764245 | 2 | 66803033 | |
| cg02788400 | 13 | 96296983 | DZIP1; DZIP1 |
| cg02860282 | 1 | 70033624 | |
| cg02899206 | 10 | 1779835 | ADARB2 |
| cg02907374 | 11 | 15095028 | CALCB |
| cg02916312 | 6 | 72596135 | RIMS1 |
| cg02948476 | 14 | 95235026 | GSC |
| cg02952008 | 1 | 228604065 | TRIM17; TRIM17; TRIM17; TRIM17 |
| cg02979001 | 14 | 105310421 | |
| cg02989521 | 11 | 107462437 | ELMOD1; LOC643923; ELMOD1 |
| cg03002846 | 10 | 135050343 | VENTX |
| cg03011535 | 20 | 42544794 | TOX2; TOX2; TOX2; TOX2; TOX2 |
| cg03018796 | 22 | 37730664 | |
| cg03020810 | 10 | 102890984 | TLX1; TLX1NB |
| cg03030717 | 12 | 65218069 | |
| cg03052869 | 10 | 88126299 | GRID1 |
| cg0305913l | 15 | 60296996 | FOXB1 |
| cg03084724 | 10 | 125853232 | |
| cg03129384 | 10 | 128994644 | FAM196A; DOCK1 |
| cg03157531 | 10 | 133795006 | BNIP3 |
| cg03168582 | 9 | 841850 | DMRT1 |
| cg03203223 | 7 | 103630549 | RELN; RELN |
| cg03238797 | 16 | 77468893 | ADAMTS18; ADAMTS18 |
| cg03242819 | 10 | 128994432 | DOCK1; FAM196A |
| cg03278146 | 18 | 5197327 | LOC642597 |
| cg03292388 | 4 | 8594514 | CPZ; CPZ; CPZ; CPZ; CPZ; CPZ |
| cg03306374 | 16 | 23847325 | PRKCB; PRKCB; PRKCB; PRKCB |
| cg03306486 | 19 | 1467952 | APC2 |
| cg03323292 | 11 | 134146132 | GLB1L3 |
| cg03356747 | 10 | 88126089 | GRID1 |
| cg03361585 | 20 | 47444241 | PREX1 |
| cg03370738 | 10 | 88126104 | GRID1 |
| cg03394150 | 16 | 28074384 | GSG1L |
| cg03401096 | 11 | 123301171 | |
| cg03405315 | 11 | 8615737 | STK33 |
| cg03437186 | 7 | 45614848 | ADCY1 |
| cg03509412 | 12 | 65515021 | WIF1; WIF1 |
| cg03559682 | 11 | 6439864 | APBB1; APBB1 |
| cg03562044 | 19 | 15342749 | EPHX3; EPHX3 |
| cg03603214 | 1 | 49242757 | AGBL4; BEND5 |
| cg03606772 | 1 | 152487856 | CRCT1 |
| cg03611452 | 19 | 38183253 | ZNF781 |
| cg03625010 | 12 | 24715484 | SOX5 |
| cg03699182 | 2 | 121104187 | INHBB |
| cg03711182 | 15 | 79383924 | RASGRF1; RASGRF1 |
| cg03735496 | 18 | 18822637 | GREB1L |
| cg03735888 | 19 | 58951602 | ZNF132 |
| cg03780132 | 5 | 42951346 | |
| cg03825010 | 5 | 159399506 | ADRA1B |
| cg03839709 | 13 | 96743492 | HS6ST3 |
| cg03867475 | 21 | 34444382 | OLIG1; OLIG1 |
| cg03884783 | 19 | 37957997 | ZNF569; ZNF569 |
| cg03921753 | 16 | 28074670 | GSG1L |
| cg04075191 | 2 | 115919785 | DPP10; DPP10; DPP10 |
| cg04100696 | 11 | 12030268 | DKK3; DKK3; DKK3 |
| cg04105282 | 10 | 99790170 | CRTAC1 |
| cg04115680 | 7 | 75889229 | SRRM3 |
| cg04123776 | 1 | 170630602 | |
| cg04172348 | 3 | 12046004 | SYN2; SYN2; SYN2; SYN2 |
| cg04182321 | 11 | 107799979 | RAB39 |
| cg04184836 | 15 | 83316640 | CPEB1; CPEB1 |
| cg04222358 | 3 | 2140256 | |
| cg04347874 | 14 | 36987408 | NKX2-1; NKX2-1 |
| cg04425632 | 7 | 137531612 | DGKI |
| cg04557544 | 16 | 12996046 | SHISA9; SHISA9 |
| cg04578774 | 11 | 44332664 | ALX4 |
| cg04578997 | 12 | 104850759 | CHST11 |
| cg04585612 | 16 | 88449996 | |
| cg04719903 | 1 | 1181956 | FAM132A |
| cg04737916 | 12 | 133196028 | P2RX2; P2RX2; P2RX2; P2RX2; P2RX2; P2RX2 |
| cg04741853 | 3 | 44037189 | |
| cg04784475 | 19 | 38183130 | ZNF781; ZNF781 |
| cg04804618 | 10 | 131761386 | EBF3 |
| cg04819760 | 10 | 22765645 | |
| cg04865110 | 4 | 6202558 | JAKMIP1; JAKMIP1 |
| cg04867733 | 1 | 77747987 | AK5; AK5; AK5 |
| cg04907523 | 1 | 213124896 | VASH2; VASH2; VASH2 |
| cg04912999 | 3 | 142682652 | PAQR9 |
| cg04922681 | 13 | 43149234 | TNFSF11; TNFSF11 |
| cg04981611 | 2 | 47798477 | KCNK12 |
| cg05033271 | 8 | 89340069 | MMP16; MMP16 |
| cg05119480 | 12 | 104850745 | CHST11 |
| cg05135549 | 22 | 17850690 | |
| cg05142211 | 3 | 181430485 | SOX2OT; SOX2 |
| cg05143123 | 5 | 136834877 | SPOCK1; SPOCK1 |
| cg05191076 | 4 | 66536186 | EPHA5; EPHA5 |
| cg05218346 | 14 | 70041283 | |
| cg05237641 | 10 | 128077307 | ADAM12; ADAM12 |
| cg05239311 | 15 | 78913147 | CHRNA3; CHRNA3; CHRNA3; CHRNA3 |
| cg05249988 | 19 | 58951684 | ZNF132 |
| cg05251676 | 5 | 38258884 | EGFLAM |
| cg05258261 | 3 | 140770608 | SPSB4 |
| cg05293775 | 8 | 33457483 | DUSP26 |
| cg05336115 | 20 | 983104 | RSPO4; RSPO4 |
| cg05347845 | 1 | 229569892 | ACTA1 |
| cg05352500 | 19 | 34972277 | WTIP |
| cg05377226 | 1 | 171810910 | DNM3; DNM3 |
| cg05446010 | 6 | 11044558 | ELOVL2; ELOVL2 |
| cg05470643 | 7 | 155579830 | |
| cg05505803 | 13 | 96296997 | DZIP1; DZIP1 |
| cg05522774 | 21 | 34443443 | OLIG1; OLIG1 |
| cg05648010 | 5 | 53545 | |
| cg05655837 | 6 | 166582188 | T |
| cg05660179 | 2 | 170218690 | LRP2 |
| cg05802452 | 1 | 228194476 | WNT3A |
| cg05804863 | 15 | 79724519 | KIAA1024 |
| cg05829782 | 14 | 74707911 | VSX2 |
| cg05841659 | 7 | 64712472 | |
| cg05849857 | 13 | 39261425 | FREM2; FREM2 |
| cg05874561 | 4 | 154709828 | SFRP2 |
| cg05916744 | 11 | 119292737 | THY1 |
| cg05924652 | 6 | 39016363 | GLP1R |
| cg05930881 | 8 | 11560540 | GATA4 |
| cg05950570 | 7 | 132261418 | PLXNA4; PLXNA4; PLXNA4 |
| cg06048524 | 10 | 44880542 | CXCL12; CXCL12; CXCL12 |
| cg06073449 | 6 | 166582310 | T |
| cg06094615 | 10 | 50887578 | C10orf53; C10orf53 |
| cg06122148 | 11 | 98891553 | CNTN5; CNTN5 |
| cg06122635 | 2 | 105461368 | |
| cg06178563 | 20 | 21494712 | NKX2-2 |
| cg06211893 | 1 | 171810778 | DNM3; DNM3; DNM3; DNM3 |
| cg06215569 | 1 | 110611465 | ALX3 |
| cg06241792 | 12 | 21680669 | C12orf39 |
| cg06274396 | 4 | 185941625 | HELT |
| cg06279276 | 16 | 67184164 | B3GNT9 |
| cg06319822 | 16 | 215960 | HBM |
| cg06399148 | 8 | 104153148 | BAALC; BAALC; C8orf56 |
| cg06401021 | 6 | 55443868 | HMGCLL1; HMGCLL1; HMGCLL1; HMGCLL1 |
| cg06445348 | 1 | 166917009 | ILDR2 |
| cg06481168 | 4 | 165878055 | C4orf39; TRIM61 |
| cg06525651 | 10 | 128994297 | FAM196A; FAM196A; DOCK1 |
| cg06554120 | 5 | 11385067 | CTNND2 |
| cg06558014 | 6 | 100051116 | |
| cg06560887 | 11 | 33890371 | LMO2; LMO2; LMO2 |
| cg06570025 | 21 | 34444245 | OLIG1; OLIG1 |
| cg06570167 | 7 | 108095719 | NRCAM; NRCAM |
| cg06573787 | 8 | 143070187 | |
| cg06577045 | 6 | 105628022 | POPDC3 |
| cg06598091 | 12 | 11653600 | |
| cg06598836 | 3 | 2140699 | |
| cg06648277 | 10 | 134600489 | NKX6-2 |
| cg06651311 | 4 | 37246230 | KIAA1239 |
| cg06664085 | 10 | 28287942 | ARMC4; ARMC4 |
| cg06668300 | 2 | 95691755 | MAL; MAL; MAL; MAL |
| cg06674731 | 2 | 154335014 | RPRM |
| cg06695611 | 2 | 180726328 | ZNF385B; MIR1258 |
| cg06740629 | 6 | 94129627 | EPHA7 |
| cg06749715 | 14 | 37132291 | PAX9 |
| cg06759058 | 6 | 28602864 | |
| cg06809252 | 1 | 110612044 | ALX3 |
| cg06862374 | 2 | 219736549 | WNT6 |
| cg06997381 | 5 | 6449090 | UBE2QL1 |
| cg07005523 | 1 | 107683187 | NTNG1; NTNG1; NTNG1 |
| cg07014673 | 2 | 237078733 | |
| cg07017374 | 13 | 28674451 | FLT3 |
| cg07028533 | 7 | 145813439 | CNTNAP2 |
| cg07028821 | 7 | 140773905 | |
| cg07057177 | 7 | 132261393 | PLXNA4; PLXNA4; PLXNA4 |
| cg07068327 | 10 | 134901279 | GPR123 |
| cg07104660 | 8 | 67873511 | |
| cg07139509 | 14 | 70038717 | C14orf162 |
| cg07155336 | 1 | 107683775 | NTNG1; NTNG1; NTNG1 |
| cg07161721 | 8 | 72459953 | |
| cg07162571 | 8 | 41624841 | ANK1; ANK1; ANK1; ANK1; ANK1 |
| cg07167168 | 20 | 41818788 | PTPRT; PTPRT |
| cg07195011 | 5 | 11904114 | CTNND2 |
| cg07254054 | 3 | 96533258 | EPHA6 |
| cg07258916 | 7 | 132262353 | PLXNA4; PLXNA4; PLXNA4 |
| cg07283114 | 8 | 65489065 | LOC401463 |
| cg07295964 | 5 | 175223982 | CPLX2 |
| cg07319626 | 2 | 207308244 | ADAM23 |
| cg07360792 | 5 | 122425702 | PRDM6 |
| cg07399369 | 3 | 142838082 | CHST2 |
| cg07413609 | 7 | 42276816 | GLI3 |
| cg07434402 | 11 | 84431535 | DLG2; DLG2 |
| cg07451080 | 3 | 44041025 | |
| cg07548607 | 2 | 187713964 | ZSWIM2 |
| cg07663789 | 5 | 32711429 | NPR3 |
| cg07680167 | 11 | 17717574 | |
| cg07690181 | 5 | 83679643 | EDIL3 |
| cg07780095 | 12 | 85874296 | ALX1 |
| cg07785314 | 20 | 61885285 | FLJ16779; NKAIN4 |
| cg07785447 | 14 | 95235127 | GSC |
| cg07821427 | 16 | 77822419 | VAT1L |
| cg07832473 | 5 | 33936253 | RXFP3 |
| cg07842403 | 3 | 134515143 | EPHB1 |
| cg07850527 | 16 | 89268040 | |
| cg07922007 | 8 | 67874858 | |
| cg07931391 | 13 | 100608263 | |
| cg07969676 | 8 | 10590641 | |
| cg07976064 | 11 | 120435056 | |
| cg07994622 | 20 | 4804052 | RASSF2 |
| cg08001895 | 6 | 94129582 | EPHA7 |
| cg08056146 | 8 | 10588013 | SOX7; SOX7 |
| cg08079580 | 10 | 133795885 | BNIP3 |
| cg08117309 | 14 | 33403862 | |
| cg08132931 | 2 | 70994962 | ADD2; ADD2; ADD2; ADD2; ADD2 |
| cg08145590 | 3 | 119422161 | C3orf15 |
| cg08154348 | 6 | 84562930 | RIPPLY2 |
| cg08157228 | 16 | 86544308 | FOXF1 |
| cg08185661 | 11 | 7273498 | SYT9 |
| cg08213098 | 1 | 35395837 | |
| cg08261094 | 7 | 37956276 | SFRP4; SFRP4 |
| cg08265644 | 14 | 70655871 | SLC8A3; SLC8A3; SLC8A3; SLC8A3 |
| cg08283882 | 8 | 25901017 | EBF2 |
| cg08315770 | 6 | 39281885 | KCNK17; KCNK17 |
| cg08322034 | 6 | 80657412 | ELOVL4 |
| cg08372619 | 8 | 109799518 | TMEM74 |
| cg08377398 | 4 | 6202553 | JAKMIP1; JAKMIP1 |
| cg08384637 | 16 | 86601133 | FOXC2 |
| cg08394412 | 16 | 50875140 | |
| cg08402652 | 11 | 94501461 | AMOTL1 |
| cg08408994 | 11 | 44330958 | ALX4 |
| cg08413157 | 20 | 41818756 | PTPRT; PTPRT |
| cg08448701 | 20 | 21686282 | PAX1 |
| cg08453926 | 13 | 27131683 | WASF3 |
| cg08458292 | 1 | 57890610 | DAB1 |
| cg08491964 | 18 | 53255771 | TCF4; TCF4; TCF4; TCF4 |
| cg08507422 | 7 | 1272512 | UNCX |
| cg08521987 | 10 | 119000927 | SLC18A2 |
| cg08568720 | 20 | 61051432 | GATA5 |
| cg08606911 | 1 | 165325231 | LMX1A |
| cg08620044 | 5 | 87980976 | LOC645323 |
| cg08632164 | 7 | 65971372 | |
| cg08663159 | 4 | 101111872 | DDIT4L |
| cg08675193 | 3 | 186080245 | DGKG; DGKG; DGKG |
| cg08675717 | 20 | 58180241 | PHACTR3 |
| cg08696727 | 10 | 28034797 | MKX |
| cg08705697 | 13 | 67804744 | PCDH9; PCDH9 |
| cg08750951 | 4 | 89378894 | HERC5 |
| cg08769966 | 15 | 92937735 | ST8SIA2 |
| cg08788717 | 11 | 8615506 | STK33 |
| cg08791131 | 16 | 58497801 | NDRG4; NDRG4; NDRG4 |
| cg08812555 | 10 | 54074788 | DKK1 |
| cg08848774 | 17 | 43047733 | |
| cg08858437 | 3 | 142838938 | CHST2; CHST2 |
| cg08870743 | 21 | 34398199 | OLIG2 |
| cg08901662 | 2 | 131595321 | |
| cg08912051 | 6 | 30227783 | HLA-L |
| cg08921126 | 20 | 10199441 | SNAP25; SNAP25 |
| cg08933939 | 6 | 100061504 | PRDM13 |
| cg08969532 | 10 | 99790438 | CRTAC1; CRTAC1 |
| cg08992305 | 4 | 165878219 | TRIM61; C4orf39 |
| cg09053680 | 10 | 135044114 | UTF1 |
| cg09088988 | 5 | 146614298 | STK32A; STK32A |
| cg09117206 | 4 | 109684077 | AGXT2L1; AGXT2L1; AGXT2L1; AGXT2L1; |
| | | | AGXT2L1; AGXT2L1; AGXT2L1; AGXT2L1 |
| cg09125812 | 8 | 41625127 | ANK1; ANK1; ANK1; ANK1; ANK1 |
| cg09141953 | 20 | 62948235 | |
| cg09150117 | 7 | 96653867 | DLX5 |
| cg09196068 | 20 | 61885270 | FLJ16779; NKAIN4 |
| cg09201151 | 15 | 84116151 | SH3GL3; SH3GL3; SH3GL3 |
| cg09225230 | 1 | 236558465 | EDARADD; EDARADD |
| cg09251429 | 11 | 124735128 | ROBO3 |
| cg09279240 | 2 | 180726252 | ZNF385B; MIR1258 |
| cg09279949 | 20 | 23030250 | THBD; THBD |
| cg09339194 | 20 | 61051585 | GATA5 |
| cg09339301 | 6 | 163836245 | QKI; QKI; QKI; QKI |
| cg09360501 | 19 | 22018958 | ZNF43; ZNF43 |
| cg09365557 | 17 | 59477564 | TBX2 |
| cg09440289 | 12 | 15475990 | PTPRO; PTPRO |
| cg09462445 | 2 | 217559131 | IGFBP5 |
| cg09493505 | 7 | 49813111 | VWC2 |
| cg09510559 | 19 | 36335144 | NPHS1 |
| cg09528825 | 16 | 28074388 | GSG1L |
| cg09553380 | 2 | 207308829 | ADAM23 |
| cg09557387 | 1 | 207818395 | CR1L |
| cg09571420 | 7 | 145813008 | CNTNAP2 |
| cg09638407 | 3 | 142839022 | CHST2; CHST2 |
| cg09639725 | 10 | 134901294 | GPR123 |
| cg09671258 | 1 | 180202530 | LHX4 |
| cg09671810 | 11 | 94501636 | AMOTL1; AMOTL1 |
| cg09684233 | 2 | 175206966 | |
| cg09754845 | 7 | 1408818 | |
| cg09767602 | 4 | 183370138 | ODZ3 |
| cg09768093 | 1 | 32930511 | ZBTB8B |
| cg09772661 | 19 | 7794952 | CLEC4G |
| cg09775312 | 10 | 93392839 | PPP1R3C; PPP1R3C |
| cg09793584 | 1 | 156391310 | C1orf61; MIR9-1 |
| cg09831026 | 12 | 133485765 | |
| cg09853371 | 4 | 57522145 | HOPX; HOPX; HOPX; HOPX; HOPX |
| cg09873164 | 1 | 152488093 | CRCT1 |
| cg09880551 | 21 | 42218932 | DSCAM; DSCAM |
| cg09935282 | 10 | 118897280 | VAX1; VAX1 |
| cg09949775 | 19 | 18902107 | COMP; COMP |
| cg09979256 | 6 | 127440104 | RSPO3; RSPO3 |
| cg09987011 | 20 | 44936040 | |
| cg09997760 | 3 | 179169556 | GNB4 |
| cg10042799 | 14 | 95236123 | GSC |
| cg10111292 | 12 | 106975112 | |
| cg10124413 | 18 | 73167919 | |
| cg10158080 | 12 | 24715864 | SOX5 |
| cg10172415 | 3 | 71803558 | GPR27; EIF4E3; EIF4E3 |
| cg10229294 | 12 | 113903210 | LHX5 |
| cg10245915 | 10 | 119001478 | SLC18A2 |
| cg10249705 | 11 | 106890150 | GUCY1A2 |
| cg10273340 | 16 | 56224793 | GNAO1; GNAO1; LOC283856 |
| cg10292139 | 8 | 97507561 | SDC2 |
| cg10325478 | 14 | 88793021 | KCNK10; KCNK10 |
| cg10356613 | 17 | 35294491 | LHX1 |
| cg10362542 | 17 | 77179709 | HRNBP3 |
| cg10364513 | 1 | 165414379 | RXRG; RXRG; RXRG; RXRG |
| cg10372047 | 5 | 63461930 | RNF180; RNF180 |
| cg10383019 | 11 | 8103101 | TUB; TUB |
| cg10457056 | 21 | 44494997 | CBS |
| cg10471437 | 16 | 28074462 | GSG1L |
| cg10507508 | 19 | 30719828 | |
| cg10512745 | 1 | 50884480 | DMRTA2 |
| cg10530851 | 14 | 37051417 | NKX2-8 |
| cg10626816 | 1 | 39025249 | |
| cg10644072 | 21 | 42218551 | DSCAM |
| cg10684547 | 7 | 155580133 | |
| cg10716835 | 10 | 94834582 | CYP26A1; CYP26A1 |
| cg10732215 | 10 | 22625465 | |
| cg10838789 | 8 | 67940989 | LRRC67 |
| cg10852177 | 7 | 84816454 | |
| cg10864596 | 5 | 178487382 | ZNF354C |
| cg10864878 | 15 | 47477004 | |
| cg10886442 | 3 | 142838320 | CHST2 |
| cg10979880 | 6 | 105584689 | BVES; BVES |
| cg11019211 | 17 | 56833043 | PPM1E |
| cg11090352 | 3 | 96532043 | EPHA6 |
| cg11092616 | 6 | 72596493 | RIMS1 |
| cg11111132 | 11 | 8041137 | |
| cg11113760 | 2 | 180726247 | ZNF385B; MIR125B |
| cg11117364 | 11 | 94501718 | AMOTL1 |
| cg11198128 | 11 | 65601332 | SNX32 |
| cg11199770 | 19 | 31841663 | TSHZ3 |
| cg11200635 | 10 | 43573195 | RET; RET |
| cg11229185 | 10 | 22625274 | |
| cg11235498 | 3 | 140771656 | SPSB4 |
| cg11253514 | 1 | 228604240 | TRIM17; TRIM17; TRIM17; TRIM17; TRIM17; |
| | | | TRIM17; TRIM17; TRIMI7 |
| cg11258089 | 5 | 59189791 | PDE4D; PDE4D |
| cg11281641 | 2 | 171674855 | GAD1; GAD1 |
| cg11342452 | 10 | 134600463 | NKX6-2 |
| cg11361827 | 17 | 45867446 | |
| cg11398452 | 10 | 118896755 | VAX1; VAX1 |
| cg11398511 | 1 | 215256635 | KCNK2; KCNK2; KCNK2; KCNK2; KCNK2 |
| cg11416384 | 4 | 37246827 | KIAA1239 |
| cg11419456 | 19 | 37157861 | ZNF461 |
| cg11514698 | 7 | 108095912 | NRCAM; NRCAM |
| cg11583963 | 19 | 53193912 | ZNF83 |
| cg11599539 | 8 | 72459614 | |
| cg11616651 | 19 | 2251837 | AMH |
| cg11630554 | 4 | 165878136 | TRIM61; C4orf39; C4orf39 |
| cg11687406 | 20 | 10199434 | SNAP25; SNAP25 |
| cg11699435 | 13 | 95365673 | SOX21 |
| cg11724516 | 9 | 115653222 | SLC46A2 |
| cg11730458 | 13 | 67805065 | PCDH9; PCDH9 |
| cg11747771 | 9 | 6645468 | GLDC |
| cg11806672 | 13 | 79176608 | POU4F1 |
| cg11821817 | 15 | 79724517 | KIAA1024 |
| cg11832210 | 10 | 91295346 | SLC16A12 |
| cg11854806 | 10 | 82116365 | DYDC2; DYDC1 |
| cg11855526 | 11 | 30607068 | MPPED2 |
| cg11880855 | 10 | 16562917 | C1QL3 |
| cg11912330 | 1 | 161228674 | PCP4L1; PCP4L1 |
| cg11980016 | 3 | 96532859 | EPHA6 |
| cg11982072 | 20 | 61051348 | GATA5 |
| cg12002303 | 15 | 68113478 | |
| cg12004183 | 7 | 71217124 | |
| cg12005098 | 10 | 91295338 | SLC16A12 |
| cg12035092 | 2 | 149633226 | KIF5C |
| cg12040830 | 11 | 112833773 | NCAM1; NCAM1; NCAM1 |
| cg12041848 | 20 | 45523294 | EYA2; EYA2; EYA2; EYA2 |
| cg12042659 | 19 | 58951599 | ZNF132 |
| cg12109566 | 6 | 30227986 | HLA-L |
| cg12147137 | 13 | 27132130 | WASF3 |
| cg12188986 | 11 | 93063886 | CCDC67; CCDC67 |
| cg12217936 | 3 | 150804058 | MED12L |
| cg12221475 | 6 | 1390622 | FOXF2 |
| cg12248614 | 19 | 41018880 | SPTBN4 |
| cg12379948 | 17 | 44896424 | WNT3 |
| cg12392473 | 20 | 48099331 | KCNB1 |
| cg12405785 | 1 | 221053409 | HLX |
| cg12473285 | 17 | 41832975 | SOST |
| cg12497564 | 3 | 139258295 | RBP1; RBP1; RBP1 |
| cg12515638 | 7 | 37956018 | SFRP4 |
| cg12573849 | 2 | 105470711 | POU3F3 |
| cg12602374 | 5 | 38557162 | LIFR; LIFR |
| cg12605662 | 18 | 56935199 | RAX |
| cg12615137 | 3 | 87040286 | VGLL3 |
| cg12623648 | 5 | 178487384 | ZNF354C |
| cg12646649 | 10 | 102987257 | LBX1 |
| cg12658947 | 11 | 132952915 | OPCML |
| cg12664209 | 20 | 13200954 | ISM1 |
| cg12686317 | 15 | 55880894 | PYGO1 |
| cg12740527 | 11 | 8289974 | |
| cg12758636 | 10 | 31609882 | ZEB1; ZEB1; ZEB1; ZEB1; ZEB1 |
| cg12824796 | 10 | 131768928 | |
| cg12859211 | 11 | 124735105 | ROBO3 |
| cg12861945 | 4 | 165878085 | C4orf39; TRIM61 |
| cg12865552 | 17 | 77721631 | |
| cg12874092 | 10 | 17271519 | VIM |
| cg12949975 | 2 | 20867352 | GDF7 |
| cg12975230 | 18 | 73167671 | |
| cg12993163 | 3 | 157821407 | SHOX2; SHOX2; SHOX2 |
| cg13012916 | 14 | 36973691 | SFTA3 |
| cg13031432 | 16 | 58497767 | NDRG4; NDRG4; NDRG4 |
| cg13042543 | 20 | 30640256 | HCK |
| cg13056495 | 7 | 134143249 | AKR1B1 |
| cg13186327 | 12 | 45444895 | DBX2 |
| cg13198321 | 15 | 58357891 | ALDH1A2; ALDH1A2; ALDH1A2; ALDH1A2 |
| cg13267264 | 8 | 70983600 | PRDM14 |
| cg13267931 | 7 | 101006308 | EMID2; EMID2 |
| cg13323701 | 6 | 118228508 | SLC35F1 |
| cg13334650 | 11 | 6440065 | APBB1; APBB1 |
| cg13346411 | 11 | 6280512 | CCKBR |
| cg13348059 | 3 | 44727069 | |
| cg13349651 | 5 | 33936307 | RXFP3 |
| cg13357482 | 6 | 105628017 | POPDC3 |
| cg13365524 | 6 | 100902093 | SIM1 |
| cg13390630 | 20 | 44803246 | CDH22 |
| cg13405332 | 17 | 19483367 | |
| cg13441730 | 9 | 10613328 | PTPRD; PTPRD |
| cg13457172 | 10 | 118897847 | VAX1; VAX1 |
| cg13459498 | 20 | 42544792 | TOX2; TOX2; TOX2; TOX2; TOX2 |
| cg13464448 | 11 | 130297513 | ADAMTS8 |
| cg13482432 | 9 | 79633350 | FOXB2 |
| cg13561592 | 9 | 118916976 | PAPPA |
| cg13562542 | 3 | 71803339 | GPR27; EIF4E3; EIF4E3 |
| cg13564825 | 19 | 38747201 | PPP1R14A |
| cg13571707 | 15 | 79383980 | RASGRF1; RASGRF1 |
| cg13603508 | 12 | 39299326 | CPNE8 |
| cg13606569 | 9 | 113341525 | SVEP1 |
| cg13632816 | 9 | 113341925 | SVEP1; SVEP1 |
| cg13689003 | 1 | 156406102 | |
| cg13703576 | 11 | 69632335 | FGF3 |
| cg13725782 | 6 | 105584718 | BVES; BVES |
| cg13742526 | 19 | 2252432 | JSRP1 |
| cg13756879 | 11 | 2161473 | INS-IGF2; IGF2AS; IGF2; IGF2; IGF2AS; IGF2 |
| cg13758646 | 9 | 101469711 | GABBR2 |
| cg13758712 | 21 | 28218959 | ADAMTS1 |
| cg13768269 | 9 | 114140 | |
| cg13776340 | 6 | 80656888 | ELOVL4 |
| cg13798146 | 15 | 83875703 | HDGFRP3 |
| cg13845982 | 20 | 61051029 | GATA5 |
| cg13913015 | 2 | 47797963 | KCNK12 |
| cg13916740 | 19 | 56904997 | ZNF582 |
| cg13928709 | 5 | 176237221 | UNC5A |
| cg13958426 | 1 | 169396637 | C1orf114; C1orf114 |
| cg13969001 | 1 | 18958084 | PAX7; PAX7; PAX7; PAX7; PAX7; PAX7 |
| cg14019323 | 12 | 65218413 | |
| cg14044640 | 7 | 27187560 | HOXA6 |
| cg14045872 | 7 | 49813065 | VWC2 |
| cg14060111 | 20 | 13200944 | ISM1 |
| cg14081924 | 3 | 142682378 | PAQR9 |
| cg14101302 | 6 | 72596557 | RIMS1 |
| cg14123923 | 13 | 79176572 | POU4F1 |
| cg14159026 | 6 | 105584551 | BVES; BVES |
| cg14168923 | 13 | 28366606 | GSX1 |
| cg14174099 | 14 | 70655862 | SLC8A3; SLC8A3; SLC8A3; SLC8A3 |
| cg14189141 | 9 | 1042605 | |
| cg14242042 | 12 | 24715250 | SOX5 |
| cg14267725 | 2 | 100721038 | AFF3; AFF3 |
| cg14314653 | 6 | 105584709 | BVES; BVES |
| cg14352983 | 18 | 6414976 | L3MBTL4 |
| cg14364356 | 21 | 44495495 | CBS |
| cg14380270 | 17 | 33700747 | SLFN11; SLFN11; SLFN11; SLFN11; SLFN11 |
| cg14409023 | 14 | 57283436 | OTX2OS1 |
| cg14414971 | 7 | 3340979 | SDK1 |
| cg14421860 | 1 | 101004934 | GPR88 |
| cg14487131 | 9 | 79633737 | FOXB2 |
| cg14556070 | 19 | 58458917 | ZNF256; ZNF256 |
| cg14568217 | 5 | 176057061 | SNCB; EIF4E1B; SNCB |
| cg14591786 | 5 | 63461574 | RNF180; RNF180 |
| cg14649650 | 10 | 13933996 | FRMD4A |
| cg14657517 | 11 | 32456910 | WT1; WIT1; WT1; WT1; WT1; WT1; WT1; WT1; WT1 |
| cg14660839 | 13 | 28674720 | FLT3; FLT3 |
| cg14667871 | 5 | 15500833 | FBXL7 |
| cg14730085 | 19 | 39522548 | FBXO27 |
| cg14730445 | 12 | 101603581 | SLC5A8 |
| cg14732324 | 5 | 528621 | |
| cg14839351 | 18 | 35146227 | BRUNOL4; BRUNOL4; BRUNOL4; BRUNOL4 |
| cg14843800 | 12 | 119419786 | SRRM4 |
| cg14866595 | 4 | 4873442 | |
| cg14896516 | 7 | 30722361 | CRHR2 |
| cg14900471 | 8 | 11561620 | GATA4 |
| cg14921743 | 20 | 44650449 | SLC12A5 |
| cg15007959 | 19 | 50931432 | SPIB |
| cg15041550 | 6 | 39016590 | GLP1R; GLP1R |
| cg15131808 | 5 | 528580 | |
| cg15139588 | 19 | 37997867 | ZNF793; ZNF793 |
| cg15146859 | 18 | 74961737 | GALR1 |
| cg15212349 | 5 | 1444852 | SLC6A3 |
| cg15221604 | 11 | 124738735 | ROBO3 |
| cg15244223 | 6 | 118228493 | SLC35F1 |
| cg15267232 | 10 | 8097689 | GATA3; GATA3 |
| cg15344220 | 11 | 8040551 | |
| cg15376615 | 5 | 153858822 | HAND1 |
| cg15409931 | 19 | 34973341 | WTIP |
| cg15431821 | 15 | 79724788 | KIAA1024 |
| cg15449956 | 8 | 106331995 | ZFPM2 |
| cg15553598 | 5 | 175792973 | ARL10 |
| cg15562912 | 3 | 140770617 | SPSB4 |
| cg15571277 | 20 | 61885249 | FLJ16779; NKAIN4 |
| cg15573040 | 15 | 92938295 | ST8SIA2 |
| cg15576900 | 1 | 44883697 | RNF220 |
| cg15607538 | 12 | 133484853 | |
| cg15613567 | 10 | 134901497 | GPR123; GPR123 |
| cg15645638 | 8 | 109799603 | TMEM74 |
| cg15654121 | 1 | 34630944 | CSMD2 |
| cg15674193 | 2 | 238535910 | LRRFIP1 |
| cg15684724 | 8 | 67875033 | |
| cg15707833 | 8 | 131455276 | |
| cg15724184 | 14 | 70346477 | SMOC1; SMOC1 |
| cg15744359 | 8 | 67940823 | LRRC67 |
| cg15760257 | 17 | 26699169 | SARM1 |
| cg15775138 | 7 | 127744389 | |
| cg15778437 | 11 | 31839521 | PAX6 |
| cg15792338 | 19 | 31840743 | TSHZ3 |
| cg15825786 | 10 | 134901297 | GPR123 |
| cg15863924 | 20 | 3388269 | C20orf194 |
| cg15898840 | 7 | 45960834 | IGFBP3; IGFBP3; IGFBP3; IGFBP3 |
| cg15919396 | 7 | 108097187 | NRCAM; NRCAM |
| cg15936446 | 5 | 42952369 | |
| cg15971888 | 8 | 67089599 | CRH |
| cg15987885 | 1 | 183386054 | NMNAT2 |
| cg15992284 | 1 | 91191254 | |
| cg15992535 | 5 | 139228150 | NRG2; NRG2; NRG2; NRG2 |
| cg15994026 | 4 | 15780306 | CD38 |
| cg16015276 | 8 | 11550445 | |
| cg16019809 | 10 | 126138593 | NKX1-2 |
| cg16041660 | 12 | 42983360 | PRICKLE1; PRICKLE1 |
| cg16043357 | 10 | 118897904 | VAX1; VAX1 |
| cg16172814 | 10 | 50969997 | OGDHL; OGDHL; OGDHL |
| cg16250461 | 6 | 30227360 | HLA-L |
| cg16276063 | 3 | 181421703 | SOX2OT |
| cg16278512 | 7 | 12443529 | VWDE; VWDE |
| cg16325777 | 10 | 43250569 | |
| cg16332610 | 15 | 74658547 | CYP11A1; CYP11A1; CYP11A1 |
| cg16400999 | 2 | 180726349 | ZNF385B; MIR1258 |
| cg16419629 | 3 | 173115536 | NLGN1 |
| cg16423505 | 13 | 92051400 | GPC5 |
| cg16468187 | 16 | 4588687 | C16orf5 |
| cg16476975 | 7 | 155164995 | |
| cg16477091 | 17 | 56833000 | PPM1E |
| cg16478774 | 6 | 132722315 | MOXD1 |
| cg16480938 | 18 | 5895225 | |
| cg16482474 | 7 | 151107637 | WDR86 |
| cg16485558 | 5 | 63461566 | RNF180; RNF180 |
| cg16499656 | 7 | 50344471 | IKZF1; IKZF1 |
| cg16501308 | 18 | 30350221 | KLHL14 |
| cg16528511 | 6 | 84562892 | RIPPLY2 |
| cg16556906 | 6 | 71666682 | B3GAT2; B3GAT2 |
| cg16557944 | 1 | 53068197 | GPX7 |
| cg16638385 | 17 | 41832753 | SOST |
| cg16647921 | 4 | 41867533 | |
| cg16697214 | 7 | 50343361 | IKZF1 |
| cg16712637 | 12 | 103352000 | ASCL1; ASCL1 |
| cg16714055 | 20 | 61051341 | GATA5 |
| cg16818740 | 20 | 4803300 | RASSF2 |
| cg16842053 | 7 | 3083333 | CARD11; CARD11 |
| cg16882226 | 2 | 101034257 | CHST10 |
| cg16910830 | 7 | 145813437 | CNTNAP2 |
| cg16915821 | 11 | 12030187 | DKK3; DKK3; DKK3 |
| cg16918905 | 2 | 220361609 | |
| cg16935295 | 8 | 97506251 | SDC2; SDC2 |
| cg16949120 | 10 | 134599783 | NKX6-2 |
| cg16958716 | 17 | 56833425 | PPM1E |
| cg16969623 | 19 | 54024182 | ZNF331; ZNF331 |
| cg17003293 | 14 | 36003826 | INSM2 |
| cg17009433 | 9 | 6645686 | GLDC; GLDC |
| cg17093995 | 7 | 49815502 | VWC2 |
| cg17188046 | 6 | 166582197 | T |
| cg17213402 | 2 | 5813650 | |
| cg17267805 | 10 | 44880545 | CXCL12; CXCL12; CXCL12 |
| cg17276590 | 10 | 119304487 | EMX2OS; EMX2; EMX2 |
| cg17307479 | 8 | 65494101 | BHLHE22 |
| cg17309441 | 8 | 48100186 | |
| cg17315500 | 12 | 103359572 | |
| cg17344755 | 3 | 154797563 | MME; MME; MME; MME |
| cg17349389 | 2 | 80530770 | CTNNA2; LRRTM1; CTNNA2 |
| cg17357285 | 6 | 28602938 | |
| cg17361203 | 11 | 117666889 | DSCAML1 |
| cg17370163 | 5 | 63461654 | RNF180; RNF180 |
| cg17418463 | 1 | 35395541 | |
| cg17429382 | 19 | 3786246 | MATK; MATK; MATK; MATK; MATK |
| cg17464383 | 5 | 122426096 | PRDM6 |
| cg17470837 | 4 | 6201430 | JAKMIP1; JAKMIP1 |
| cg17498296 | 10 | 124907540 | HMX2 |
| cg17512353 | 6 | 30227802 | HLA-L |
| cg17535595 | 13 | 53422808 | PCDH8; PCDH8 |
| cg17565078 | 13 | 27334723 | GPR12; GPR12 |
| cg17567560 | 10 | 105036863 | INA |
| cg17627617 | 3 | 142682682 | PAQR9 |
| cg17688525 | 18 | 6414978 | L3MBTL4 |
| cg17714276 | 4 | 156588619 | GUCY1A3; GUCY1A3; GUCY1A3; GUCY1A3; |
| | | | GUCY1A3; GUCY1A3; GUCY1A3 |
| cg17721710 | 1 | 220101698 | SLC30A10 |
| cg17757602 | 5 | 42952113 | |
| cg17815538 | 2 | 238536315 | LRRFIP1 |
| cg17839237 | 7 | 19157193 | TWIST1; TWIST1 |
| cg17859110 | 20 | 41818770 | PTPRT; PTPRT |
| cg17868340 | 10 | 125853676 | |
| cg17898329 | 4 | 154713496 | |
| cg17994139 | 7 | 27187556 | HOXA6 |
| cg17996619 | 10 | 134600701 | NKX6-2 |
| cg18013519 | 4 | 6473964 | PPP2R2C; PPP2R2C |
| cg18035229 | 8 | 70984270 | PRDM14 |
| cg18066271 | 5 | 100236783 | ST8SIA4; ST8SIA4 |
| cg18120376 | 4 | 142054254 | RNF150; RNF150 |
| cg18174928 | 5 | 38557085 | LIFR; LIFR |
| cg18180569 | 1 | 44883362 | RNF220 |
| cg18206027 | 7 | 49813486 | VWC2; VWC2 |
| cg18249634 | 3 | 32860467 | TRIM71 |
| cg18276638 | 1 | 214360619 | |
| cg18278265 | 20 | 61051438 | GATA5 |
| cg18290848 | 7 | 89950671 | |
| cg18313899 | 6 | 118228498 | SLC35F1 |
| cg18314424 | 4 | 184718605 | |
| cg18326657 | 6 | 30227729 | HLA-L |
| cg18402615 | 10 | 124896659 | HMX3 |
| cg18412834 | 20 | 61885291 | FLJ16779; NKAIN4 |
| cg18420512 | 7 | 128828982 | SMO; SMO |
| cg18428688 | 19 | 58609744 | ZSCAN18; ZSCAN18; ZSCAN18; ZSCAN18 |
| cg18488855 | 14 | 27066634 | NOVA1; NOVA1; NOVA1 |
| cg18552861 | 2 | 20865845 | GDF7 |
| cg18560328 | 11 | 7273148 | SYT9 |
| cg18603228 | 3 | 13590439 | FBLN2; FBLN2 |
| cg18607529 | 7 | 50343869 | IKZF1 |
| cg18623980 | 2 | 45240563 | |
| cg18624900 | 10 | 91295643 | SLC16A12 |
| cg18646207 | 10 | 118897836 | VAX1; VAX1 |
| cg18657094 | 3 | 2140030 | |
| cg18689958 | 5 | 33936232 | RXFP3 |
| cg18710929 | 6 | 118228078 | SLC35F1 |
| cg18723978 | 7 | 42276814 | GLI3 |
| cg18749015 | 6 | 45631363 | |
| cg18781240 | 13 | 27131676 | WASF3 |
| cg18786873 | 1 | 110610899 | ALX3 |
| cg18794839 | 1 | 215256262 | KCNK2; KCNK2; KCNK2 |
| cg18800085 | 1 | 167599719 | RCSD1 |
| cg18881684 | 1 | 70033592 | |
| cg18920423 | 10 | 23481389 | PTF1A |
| cg18935813 | 1 | 111149153 | KCNA2 |
| cg18972849 | 4 | 134067789 | |
| cg18991611 | 8 | 49468828 | |
| cg18996590 | 8 | 30890583 | WRN; PURG; PURG; PURG |
| cg19054524 | 20 | 21686273 | PAX1 |
| cg19068510 | 13 | 100642106 | |
| cg19079194 | 2 | 213403321 | ERBB4; ERBB4; ERBB4; ERBB4 |
| cg19118812 | 7 | 37488438 | ELMO1; ELMO1 |
| cg19126300 | 11 | 32457162 | WT1; WIT1; WT1; WT1; WT1 |
| cg19186145 | 2 | 45169562 | SIX3 |
| cg19194098 | 10 | 28287983 | ARMC4 |
| cg19206040 | 1 | 37500441 | GRIK3 |
| cg19241327 | 7 | 157484559 | PTPRN2; PTPRN2; PTPRN2 |
| cg19266910 | 11 | 107462445 | ELMOD1; LOC643923; ELMOD1 |
| cg19320476 | 11 | 82443592 | FAM181B |
| cg19355087 | 10 | 134600295 | NKX6-2 |
| cg19412467 | 2 | 107502679 | ST6GAL2; ST6GAL2; ST6GAL2 |
| cg19427610 | 12 | 65515031 | WIF1; WIF1 |
| cg19443257 | 3 | 140770549 | SPSB4 |
| cg19461621 | 18 | 500979 | COLEC12 |
| cg19485202 | 19 | 18902081 | COMP; COMP |
| cg19509715 | 3 | 44037128 | |
| cg19544662 | 2 | 88752056 | FOXI3 |
| cg19570244 | 11 | 32457158 | WT1; WIT1; WT1; WT1; WT1 |
| cg19584875 | 14 | 90528213 | KCNK13; KCNK13 |
| cg19589811 | 11 | 82443961 | FAM181B |
| cg19594305 | 19 | 34112991 | CHST8; CHST8; CHST8; CHST8 |
| cg19618483 | 5 | 32711355 | NPR3 |
| cg19655456 | 19 | 37959961 | ZNF570 |
| cg19665362 | 15 | 83316838 | CPEB1 |
| cg19721867 | 10 | 119001066 | SLC18A2 |
| cg19734015 | 7 | 94284432 | SGCE; SGCE; PEG10; SGCE; PEG10 |
| cg19736503 | 6 | 84563604 | RIPPLY2 |
| cg19752627 | 7 | 98467380 | TMEM130; TMEM130; TMEM130 |
| cg19761848 | 2 | 237076815 | GBX2 |
| cg19831575 | 11 | 69590090 | FGF4; FGF4 |
| cg19850348 | 15 | 55880997 | PYGO1 |
| cg19935171 | 2 | 39893078 | TMEM178; TMEM178; TMEM178 |
| cg19950455 | 21 | 44495595 | CBS |
| cg20012008 | 7 | 69062534 | AUTS2; AUTS2; AUTS2 |
| cg20014049 | 10 | 23481385 | PTF1A |
| cg20052751 | 3 | 157155332 | VEPH1; VEPH1; PTX3; VEPH1 |
| cg20078466 | 7 | 50344331 | IKZF1 |
| cg20129213 | 8 | 104512317 | RIMS2 |
| cg20162381 | 12 | 3310097 | TSPAN9; TSPAN9 |
| cg20183619 | 7 | 155241490 | |
| cg20193324 | 13 | 26626134 | SHISA2 |
| cg20213228 | 20 | 61810348 | |
| cg20232102 | 19 | 38747174 | PPP1R14A |
| cg20250080 | 17 | 66596999 | FAM20A; FAM20A |
| cg20265733 | 20 | 61051032 | GATA5 |
| cg20298273 | 1 | 156897330 | C1orf92 |
| cg20311863 | 1 | 91184126 | BARHL2 |
| cg20318608 | 1 | 179712591 | FAM163A |
| cg20339230 | 15 | 92937360 | ST8SIA2 |
| cg20340508 | 21 | 34442377 | OLIG1 |
| cg20347882 | 7 | 155166554 | |
| cg20478129 | 14 | 27067372 | NOVA1; NOVA1; NOVA1 |
| cg20483857 | 2 | 107502677 | ST6GAL2; ST6GAL2; ST6GAL2 |
| cg20498414 | 20 | 43439400 | RIMS4 |
| cg20541723 | 3 | 85007894 | CADM2; CADM2 |
| cg20560075 | 5 | 146257484 | PPP2R2B; PPP2R2B; PPP2R2B; PPP2R2B; |
| | | | PPP2R2B; PPP2R2B; PPP2R2B |
| cg20648847 | 11 | 66326767 | ACTN3 |
| cg20649951 | 10 | 88126306 | GRID1 |
| cg20656261 | 20 | 4803864 | RASSF2 |
| cg20737185 | 6 | 110679566 | C6orf186 |
| cg20740029 | 12 | 101603835 | SLC5A8; SLC5A8 |
| cg20761860 | 5 | 131992114 | |
| cg20771178 | 11 | 8615675 | STK33 |
| cg20788479 | 3 | 179169536 | GNB4 |
| cg20842253 | 2 | 27529788 | TRIM54; TRIM54 |
| cg20870512 | 7 | 1272515 | UNCX |
| cg20893717 | 7 | 100318190 | EPO |
| cg20930060 | 6 | 163835395 | QKI; QKI; QKI; QKI |
| cg20979852 | 3 | 140770683 | SPSB4 |
| cg20987924 | 15 | 60296981 | FOXB1 |
| cg21039708 | 14 | 57278729 | OTX2OS1 |
| cg21067341 | 12 | 96883381 | |
| cg21068911 | 11 | 107462430 | ELMOD1; LOC643923; ELMOD1 |
| cg21121136 | 3 | 14852327 | |
| cg21145136 | 15 | 84116115 | SH3GL3; SH3GL3; SH3GL3 |
| cg21173447 | 21 | 42218964 | DSCAM; DSCAM |
| cg21189727 | 8 | 67940950 | LRRC67 |
| cg21240762 | 8 | 67089388 | CRH |
| cg21258057 | 20 | 61885262 | FLJ16779; NKAIN4 |
| cg21338532 | 2 | 154335348 | RPRM |
| cg21401219 | 3 | 150803669 | MED12L |
| cg21401879 | 2 | 45162036 | |
| cg21426003 | 2 | 237076811 | GBX2 |
| cg21521683 | 12 | 22487682 | ST8SIA1 |
| cg21548032 | 12 | 104850767 | CHST11 |
| cg21552709 | 6 | 94129636 | EPHA7 |
| cg21553524 | 2 | 39893185 | TMEM178; TMEM178 |
| cg21578219 | 1 | 18434542 | IGSF21; IGSF21 |
| cg21591173 | 7 | 69063475 | AUTS2; AUTS2; AUTS2 |
| cg21653184 | 19 | 58609361 | ZSCAN18; ZSCAN18; ZSCAN18, ZSCAN18; ZSCAN18 |
| cg21667878 | 11 | 2160980 | INS-IGF2; IGF2AS; IGF2; IGF2; IGF2AS; IGF2 |
| cg21692846 | 11 | 134146075 | GLB1L3 |
| cg21787291 | 6 | 124125170 | NKAIN2; NKAIN2 |
| cg21802055 | 4 | 4868794 | |
| cg22007163 | 6 | 28602927 | |
| cg22007227 | 1 | 32930544 | ZBTB8B |
| cg22028075 | 4 | 6564820 | |
| cg22031998 | 19 | 58609730 | ZSCAN18; ZSCAN18; ZSCAN18; ZSCAN18 |
| cg22091110 | 18 | 5630348 | |
| cg22111078 | 5 | 172655925 | |
| cg22152407 | 20 | 23030446 | THBD |
| cg22212691 | 2 | 220417868 | OBSL1 |
| cg22263131 | 6 | 94129697 | EPHA7 |
| cg22276619 | 8 | 21645332 | GFRA2; GFRA2; GFRA2 |
| cg22284043 | 13 | 92051576 | GPC5 |
| cg22286978 | 19 | 58858806 | A1BG |
| cg22298430 | 17 | 5404330 | LOC728392; LOC728392 |
| cg22321089 | 13 | 21649722 | |
| cg22371972 | 11 | 22364961 | SLC17A6 |
| cg22413388 | 22 | 46367617 | WNT7B |
| cg22459630 | 15 | 68121028 | LBXCOR1 |
| cg22474464 | 20 | 21492914 | NKX2-2 |
| cg22490134 | 10 | 22624847 | |
| cg22546168 | 10 | 135050326 | VENTX |
| cg22604123 | 1 | 32930522 | ZBTB8B |
| cg22610211 | 3 | 139654264 | CLSTN2 |
| cg22623967 | 3 | 16554910 | RFTN1 |
| cg22630755 | 5 | 134871807 | NEUROG1 |
| cg22657780 | 2 | 180725907 | ZNF385B; MIR1258; ZNF385B |
| cg22675486 | 1 | 165414544 | RXRG; RXRG |
| cg22723056 | 11 | 7273378 | SYT9; SYT9 |
| cg22746058 | 10 | 23481176 | PTF1A |
| cg22830113 | 2 | 127783168 | |
| cg22863523 | 11 | 14995201 | CALCA; CALCA; CALCA |
| cg22871002 | 14 | 62279623 | |
| cg22876812 | 2 | 71116188 | |
| cg22882665 | 2 | 45241110 | |
| cg22901008 | 10 | 91295650 | SLC16A12 |
| cg22902266 | 9 | 96714313 | BARX1 |
| cg22903300 | 6 | 29760765 | HCG4 |
| cg22931182 | 11 | 44331623 | ALX4; ALX4 |
| cg22937649 | 17 | 8926794 | NTN1 |
| cg22955973 | 3 | 154797947 | MME; MME; MME; MME |
| cg23010538 | 1 | 190447290 | FAM5C |
| cg23018873 | 11 | 94501467 | AMOTL1 |
| cg23020486 | 1 | 236558874 | EDARADD; EDARADD; EDARADD |
| cg23027580 | 8 | 67089513 | CRH |
| cg23048481 | 1 | 39044196 | |
| cg23080354 | 22 | 45405880 | PHF21B; PHF21B |
| cg23154059 | 2 | 219736250 | WNT6 |
| cg23194354 | 7 | 3340459 | SDK1 |
| cg23236554 | 5 | 132947501 | FSTL4 |
| cg23242697 | 8 | 93115324 | |
| cg23250494 | 15 | 78913474 | CHRNA3; CHRNA3; CHRNA3; CHRNA3 |
| cg23253569 | 21 | 34398222 | OLIG2 |
| cg23272632 | 14 | 77737146 | NGB |
| cg23291854 | 14 | 88792913 | KCNK10; KCNK10 |
| cg23333915 | 13 | 95365509 | SOX21 |
| cg23363014 | 6 | 30227800 | HLA-L |
| cg23391785 | 1 | 171810972 | DNM3; DNM3 |
| cg23415756 | 17 | 8925752 | NTN1 |
| cg23452969 | 11 | 62691182 | |
| cg23477406 | 12 | 132312741 | MMP17 |
| cg23502778 | 14 | 27068135 | NOVA1; NOVA1; NOVA1 |
| cg23609571 | 14 | 70655845 | SLC8A3; SLC8A3; SLC8A3; SLC8A3 |
| cg23688510 | 6 | 166581929 | T; T |
| cg23708361 | 7 | 145813432 | CNTNAP2 |
| cg23720732 | 20 | 44650380 | SLC12A5; SLC12A5 |
| cg23721712 | 6 | 30227967 | HLA-L |
| cg23770904 | 20 | 61051561 | GATA5 |
| cg23808946 | 2 | 45169548 | SIX3 |
| cg23811464 | 12 | 24716204 | SOX5 |
| cg23906738 | 14 | 36987301 | NKX2-1; NKX2-1 |
| cg23936023 | 5 | 82769030 | VCAN; VCAN; VCAN; VCAN |
| cg23991622 | 10 | 17271303 | VIM |
| cg24034005 | 14 | 97059192 | |
| cg24037897 | 10 | 16562626 | C1QL3 |
| cg24087887 | 13 | 39261432 | FREM2; FREM2 |
| cg24094550 | 12 | 54333565 | HOXC13 |
| cg24122124 | 19 | 5339092 | PTPRS; PTPRS; PTPRS; PTPRS |
| cg24319171 | 12 | 187480 | IQSEC3; IQSEC3 |
| cg24320612 | 20 | 61051317 | GATA5 |
| cg24342409 | 15 | 92937992 | ST8SIA2 |
| cg24359323 | 11 | 69634372 | FGF3 |
| cg24395801 | 1 | 86622624 | COL24A1 |
| cg24400921 | 12 | 42984337 | PRICKLE1 |
| cg24446548 | 7 | 19157263 | TWIST1; TWIST1 |
| cg24453580 | 12 | 49691064 | PRPH |
| cg24500900 | 20 | 61051423 | GATA5 |
| cg24534742 | 13 | 43149281 | TNFSF11; TNFSF11 |
| cg24565369 | 7 | 119913683 | KCND2 |
| cg24610236 | 7 | 30722114 | CRHR2; CRHR2 |
| cg24617696 | 2 | 121104312 | INHBB |
| cg24632241 | 2 | 80530431 | CTNNA2; LRRTM1; CTNNA2 |
| cg24642320 | 4 | 186049687 | |
| cg24662718 | 1 | 108507468 | VAV3 |
| cg24678137 | 11 | 7273735 | SYT9 |
| cg24686074 | 10 | 102497666 | |
| cg24761507 | 17 | 35293930 | LHX1 |
| cg24767148 | 10 | 23481786 | PTF1A |
| cg24792289 | 5 | 134825895 | |
| cg24805239 | 7 | 24797486 | DFNA5; DFNA5; DFNA5; DFNA5; DFNA5 |
| cg24809973 | 8 | 72468820 | |
| cg24876960 | 5 | 1883214 | IRX4 |
| cg24879782 | 2 | 19561482 | |
| cg24880701 | 15 | 68121563 | LBXCOR1 |
| cg24886267 | 5 | 167956306 | FBLL1 |
| cg24890043 | 2 | 135476297 | TMEM163 |
| cg24908814 | 5 | 132947725 | FSTL4 |
| cg24937747 | 5 | 1882902 | IRX4 |
| cg24979348 | 12 | 15374303 | RERG; RERG |
| cg24989739 | 6 | 105628027 | POPDC3 |
| cg25014318 | 6 | 39016776 | GLP1R |
| cg25019648 | 17 | 72353261 | BTBD17 |
| cg25020286 | 1 | 18956947 | PAX7; PAX7; PAX7 |
| cg25075147 | 2 | 175547399 | WIPF1 |
| cg25082959 | 13 | 103046930 | FGF14 |
| cg25104105 | 1 | 14925198 | KIAA1026; KIAA1026 |
| cg25146017 | 6 | 110679400 | C6orf186 |
| cg25167643 | 7 | 121513538 | PTPRZ1; PTPRZ1 |
| cg25185881 | 4 | 156681047 | GUCY1B3 |
| cg25303599 | 11 | 61595807 | FADS2; FADS2 |
| cg25333258 | 7 | 44365029 | CAMK2B; CAMK2B; CAMK2B; CAMK2B; CAMK2B; CAMK2B; |
| | | | CAMK2B; CAMK2B; CAMK2B; CAMK2B; CAMK2B; CAMK2B; |
| | | | CAMK2B; CAMK2B; CAMK2B; CAMK2B |
| cg25402610 | 7 | 127807500 | |
| cg25446309 | 10 | 131770223 | |
| cg25485192 | 7 | 94284439 | SGCE; SGCE; PEG10; SGCE; PEG10 |
| cg25531700 | 12 | 132312744 | MMP17 |
| cg25561581 | 22 | 45405899 | PHF21B; PHF21B |
| cg25640822 | 5 | 134871645 | NEUROG1 |
| cg25649038 | 6 | 6546777 | LOC285780 |
| cg25662463 | 10 | 118897857 | VAX1; VAX1 |
| cg25669309 | 4 | 184826324 | STOX2 |
| cg25670060 | 11 | 98891544 | CNTN5; CNTN5 |
| cg25670330 | 3 | 138665984 | FOXL2; C3orf72 |
| cg25681339 | 17 | 1174148 | BHLHA9 |
| cg25723050 | 1 | 224804222 | CNIH3; CNIH3 |
| cg25730685 | 1 | 2375010 | |
| cg25796439 | 20 | 13200939 | ISM1 |
| cg25805709 | 3 | 44596770 | ZNF167; ZNF167; ZNF167; ZNF167 |
| cg25830696 | 17 | 42030479 | PYY |
| cg25848557 | 1 | 108507766 | VAV3 |
| cg25875213 | 19 | 38183055 | ZNF781; ZNF781 |
| cg25905674 | 17 | 43047856 | |
| cg25920406 | 10 | 35929369 | FZD8 |
| cg25927708 | 2 | 119603813 | EN1 |
| cg25932164 | 20 | 37434950 | PPP1R16B |
| cg25942031 | 5 | 178487410 | ZNF354C |
| cg25964032 | 10 | 35929326 | FZD8 |
| cg25975712 | 22 | 48971051 | FAM19A5; FAM19A5 |
| cg26000619 | 19 | 2251067 | AMH |
| cg26000663 | 11 | 26353723 | ANO3; ANO3 |
| cg26043257 | 4 | 15780238 | CD38 |
| cg26068551 | 1 | 57111117 | PRKAA2 |
| cg26072058 | 1 | 91191055 | |
| cg26110710 | 13 | 88323607 | SLITRK5 |
| cg26114043 | 4 | 128544375 | |
| cg26149275 | 2 | 176950007 | EVX2 |
| cg26150071 | 1 | 34629559 | CSMD2 |
| cg26170805 | 18 | 5630016 | |
| cg26195812 | 2 | 27071332 | DPYSL5 |
| cg26224671 | 13 | 43148935 | TNFSF11; TNFSF11 |
| cg26261793 | 1 | 171810543 | DNM3; DNM3 |
| cg26271891 | 2 | 180726249 | ZNF385B; MIR1258 |
| cg26339504 | 10 | 118897859 | VAX1; VAX1 |
| cg26365854 | 11 | 44330903 | ALX4 |
| cg26432256 | 14 | 77607222 | ZDHHC22 |
| cg26466094 | 1 | 77748129 | AK5; AK5 |
| cg26477488 | 5 | 153858936 | HAND1 |
| cg26477573 | 19 | 15342915 | EPHX3; EPHX3 |
| cg26532358 | 6 | 118228871 | SLC35F1; SLC35F1 |
| cg26540367 | 16 | 215864 | HBM |
| cg26541867 | 4 | 37246688 | KIAA1239 |
| cg26565021 | 19 | 34112825 | CHST8; CHST8 |
| cg26599006 | 22 | 19137371 | GSC2 |
| cg26608883 | 11 | 15095024 | CALCB |
| cg26649384 | 2 | 154334651 | RPRM; RPRM |
| cg26659805 | 19 | 2251588 | AMH |
| cg26678605 | 10 | 118925011 | |
| cg26692294 | 5 | 6449001 | UBE2QL1; UBE2QL1 |
| cg26705425 | 19 | 54024076 | ZNF331 |
| cg26708235 | 13 | 25946397 | ATPBA2 |
| cg26721193 | 8 | 93114951 | |
| cg26721264 | 18 | 74961727 | GALR1 |
| cg26733786 | 12 | 65515034 | WIF1; WIF1 |
| cg26756083 | 8 | 89339622 | MMP16; MMP16; MMP16; MMP16 |
| cg26770917 | 21 | 34444339 | OLIG1; OLIG1 |
| cg26818735 | 7 | 19156621 | TWIST1 |
| cg26831241 | 2 | 175546916 | WIPF1 |
| cg26844246 | 5 | 170736277 | TLX3 |
| cg26886381 | 13 | 96296979 | DZIP1; DZIP1 |
| cg26961808 | 7 | 128470913 | FLNC; FLNC |
| cg26976732 | 16 | 216100 | HBM |
| cg26985666 | 11 | 35441088 | SLC1A2; SLC1A2 |
| cg26986911 | 5 | 33936292 | RXFP3 |
| cg26988895 | 7 | 6576353 | GRID2IP |
| cg27034576 | 10 | 118031654 | GFRA1; GFRA1; GFRA1; GFRA1 |
| cg27037018 | 5 | 1445567 | SLC6A3 |
| cg27058257 | 19 | 30019529 | VSTM2B |
| cg27066284 | 17 | 71161258 | SSTR2; SSTR2 |
| cg27101125 | 19 | 17392770 | ANKLE1 |
| cg27125849 | 17 | 59473674 | |
| cg27205687 | 11 | 106888787 | GUCY1A2; GUCY1A2 |
| cg27237300 | 21 | 34442292 | OLIG1 |
| cg27304110 | 1 | 114695695 | SYT6 |
| cg27316886 | 12 | 3600106 | PRMT8 |
| cg27398263 | 13 | 79177700 | POU4F1 |
| cg27420520 | 12 | 103352267 | ASCL1 |
| cg27464184 | 12 | 114075881 | |
| cg27486637 | 4 | 176987174 | WDR17; WDR17; WDR17; WDR17 |
| cg27493301 | 12 | 42982929 | PRICKLE1 |
| cg27510832 | 1 | 76080684 | |
| cg27511255 | 20 | 47444851 | PREX1 |
| cg27545919 | 19 | 37157995 | ZNF461 |
| cg27547954 | 1 | 76081962 | |
| cg27591450 | 17 | 75525004 | |
| cg27605748 | 5 | 42951711 | |
| cg27606567 | 3 | 44040811 | |
| cg27628784 | 10 | 131767387 | |
| cg27648738 | 15 | 84115811 | SH3GL3; SH3GL3 |
| cg27649239 | 15 | 68120393 | LBXCOR1 |

### Example 8: Narrowing Probes in Two Cohorts and Verification

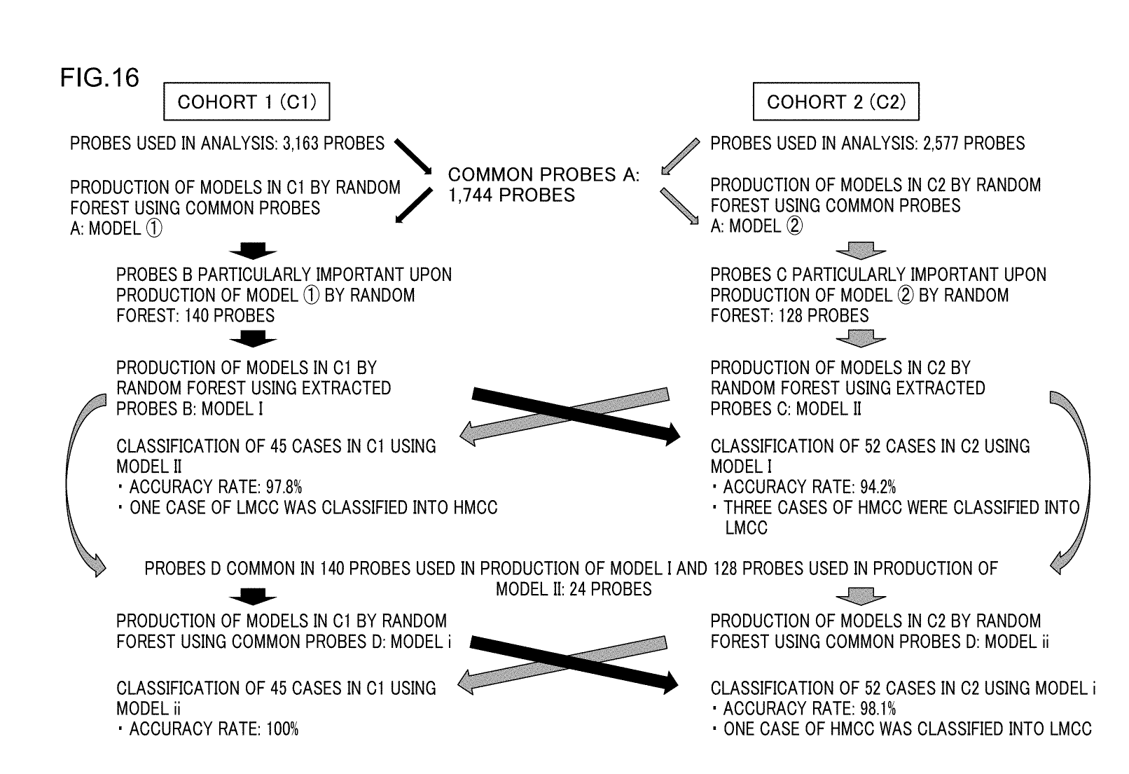

The patient groups of Examples 1 and 2 were defined as Cohort 1 (C1) and Cohort 2 (C2), respectively, and the narrowing of probes to be used in analysis and the verification thereof were carried out according to the following procedures (FIG. 16).

1) First, using the algorithm called Random Forest, prediction models regarding classification into HMCC and LMCC were produced.
2) From 3,163 probes extracted from Cohort 1 and 2,577 probes extracted from Cohort 2, 1,744 probes common in the two cohorts were extracted.
3) Using the extracted 1,744 probes, models were produced in C1 by performing Random Forest, and the classification results of C2 were then predicted.
4) Using the extracted 1,744 probes, models were produced in C2 by performing Random Forest, and the classification results of C1 were then predicted.
5) In the above 3) and 4), the importance of variables used in the production of models by Random Forests was confirmed, and such variables were narrowed to 0.002 or more.
6) As a result of the above 5), 140 probes were extracted from the C1 models and 128 probes were extracted from the C2 models.
7) In the above 6), when the common probes were extracted, 24 probes remained.
8) Using these 24 probes, the above predictions 3) and 4) were carried out.
8-1) When models were produced in C1 and the classification results of C2 were then predicted, the accuracy rate was found to be 98.1% (the answer was different from the correct answer only in one case).
8-2) When models were produced in C2 and the classification results of C1 were then predicted, the accuracy rate was found to be 100%.

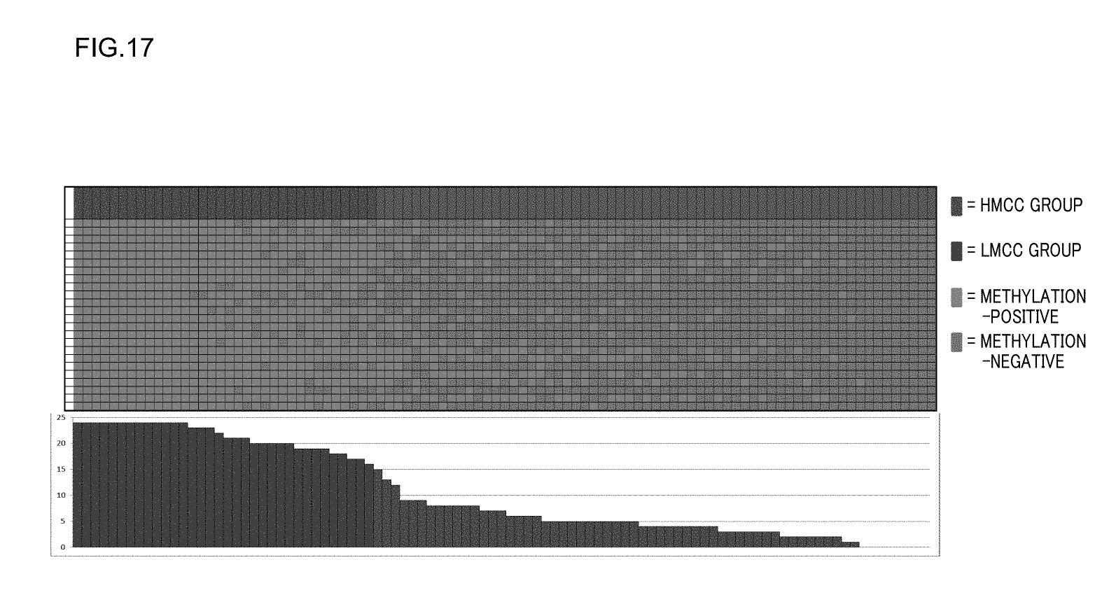

The extracted 24 probes are shown in Table 8. Using the 24 probes, the conditions shown in the slide were determined, and the 97 cases used in the analysis were classified again. The obtained results are shown in FIG. 17. In the present classification, when each probe has a β value of 0.5 or more, it was determined that the probe was methylation-positive. Moreover, among the 24 probes, when the number of methylation-positive probes was 16 or more, it was determined that the case was among an HMCC group, and when the number of methylation-positive probes was 15 or less, it was determined that the case was among an LMCC group.

TABLE 8
Information of 24 genes

| | | Chromosome | Location |
|---|---|---|---|
| TargetID | UCSC_REFGENE_NAME | No. (chr) | information |
| cg01791410 | | 3 | 150802997 |
| cg01802453 | ODZ3 | 4 | 183370148 |
| cg02484469 | GATA5 | 20 | 61051036 |
| cg02916312 | RIMS1 | 6 | 72596135 |
| cg03839709 | HS6ST3 | 13 | 96743492 |
| cg05218346 | | 14 | 70041283 |
| cg07005523 | NTNG1 | 1 | 107683187 |
| cg07068327 | GPR123 | 10 | 134901279 |
| cg07258916 | PLXNA4 | 7 | 132262353 |
| cg07360792 | PRDM6 | 5 | 122425702 |
| cg09767602 | ODZ3 | 4 | 183370138 |
| cg11092616 | RIMS1 | 6 | 72596493 |
| cg12646649 | LBX1 | 10 | 102987257 |
| cg13267931 | EMID2 | 7 | 101006308 |
| cg16041660 | PRICKLE1 | 12 | 42983360 |
| cg16958716 | PPM1E | 17 | 56833425 |
| cg17188046 | | 6 | 166582197 |
| cg18412834 | FLJ16779; NKAIN4 | 20 | 61885291 |
| cg20012008 | AUTS2 | 7 | 69062534 |
| cg20265733 | GATA5 | 20 | 61051032 |
| cg20339230 | ST8SIA2 | 15 | 92937360 |
| cg21787291 | NKAIN2 | 6 | 124125170 |
| cg24792289 | | 5 | 134825895 |
| cg27628784 | | 10 | 131767387 |

* As a “reference” gene of location information, “hg19” was used.

In the table, each gene is specified with chromosome number and location information.

For example, when the chromosome number is 3 and location information is 150802997, it indicates that one specific nucleotide present at 150802997 of chromosome 3 has been methylated. Methylation in the present classification means that “one nucleotide in a specific site existing on the human genome has been methylated.”

Using the models produced in one cohort, the other cohort was classified. As a result, the accuracy rate was more than 90% in both of the cohorts. Accordingly, it was considered that the reproducibility of classification in each cohort is high, and that variables (probes) used in the classification of the two cohorts are constituted with those having similar tendency. Moreover, among the probes used for the models produced in each cohort, the common 24 probes were used, and models were produced again in each cohort by performing random forest. Thereafter, the other cohort was classified. As a result, all cases, except for one case, were accurately classified.

From the aforementioned results, it was demonstrated that, by using the extracted 24 probes, classification into HMCC or LMCC can be carried out with precision almost equivalent to the case of using 3,144 or 2,577 probes.

That is to say, it was demonstrated that conversion to a simple detection system, which is directed towards clinical application, is possible.

### INDUSTRIAL APPLICABILITY

The method of the present invention has a small variation in the results caused by specimen collection conditions, and thus, even using a specimen collected upon excision of a primary lesion, the results equivalent to those of methylation profiles in the tumor at the time point of initiation of the treatment were obtained. Moreover, since the method of the present invention enables not only selection of a primary treatment and a secondary treatment in a combination therapy, but also determination of the suitability of the order in which these therapies are applied, the present method can provide the optimal therapeutic planning depending on the conditions of a patient or disease. That is to say, according to the present invention, the responsiveness of a patient to cancer drug therapy can be predicted with high precision, economical and/or physical burden on the patient are reduced, and administration guidelines with higher cost-effectiveness can be provided.

All publications, patents and patent applications cited in the present description are incorporated herein by reference in their entirety.

1. A method for predicting the responsiveness of a colorectal cancer patient to cancer drug therapy using an anti-EGFR antibody, the method comprising:
(1) a step of measuring a level of DNA methylation in a specimen comprising a colorectal cancer tissue, colorectal cancer cells, or colorectal cancer cell-derived DNA of a subject,
(2) a step of defining a gene having a β value of 0.5 or more as methylation positive, and then, classifying the subject into a highly-methylated group when the ratio of a methylation-positive gene is 60% or more, and classifying the subject into a low-methylated group when the ratio of a methylation-positive gene is less than 60%, and
(3) a step of determining that the subject is sensitive to an anti-EGFR antibody when the subject is classified into the low-methylated group, and determining that the subject is resistant to an anti-EGFR antibody when the subject is classified into the highly-methylated group, wherein
the analysis is carried out on at least 4 or more marker genes, as targets, selected from the 24 genes shown in Table 8, or CACNA1G, LOX, SLC30A10, ELMO1, HAND1, IBN2 and THBD.

2-4. (canceled)

5. The method according to claim 1, wherein the analysis is carried out on 4 to 20 marker genes, as targets, selected from the 24 genes shown in Table 8, or CACNA1G, LOX, SLC30A10, ELMO1, HAND1, IBN2 and THBD.

6. The method according to claim 1, wherein the analysis is carried out on 4 to 10 marker genes, as targets, selected from the 24 genes shown in Table 8, or CACNA1G, LOX, SLC30A10, ELMO1, HAND1, IBN2 and THBD.

7. The method according to claim 1, wherein the marker genes are the 24 genes shown in Table 8.

8-10. (canceled)

11. The method according to claim 1, wherein the suitability of the order of cancer drug therapies can be determined.

12. A probe set for predicting the responsiveness of a colorectal cancer patient to cancer drug therapy, wherein
the probe set comprises a probe which comprises a sequence complementary to a region comprising a CpG site of at least one of 4 or more marker genes selected from the 24 genes shown in Table 8, or CACNA1G, LOX, SLC30A10, ELMO1, HAND1, IBN2 and THBD, and which is capable of detecting the presence or absence of the methylation of the CpG site.

13. The probe set according to claim 12, wherein the marker genes are the 24 genes shown in Table 8.

14. A kit for predicting the responsiveness of a colorectal cancer patient to cancer drug therapy, wherein the kit comprises:
(a) a probe which comprises a sequence complementary to a region comprising a CpG site of at least one of 4 or more marker genes selected from the 24 genes shown in Table 8, or CACNA1G, LOX, SLC30A10, ELMO1, HAND1, IBN2 and THBD, and which is capable of detecting the presence or absence of the methylation of the CpG site, and
(b) a primer pair which binds to a region comprising a CpG site of at least one of 4 or more marker genes selected from the 24 genes shown in Table 8, or CACNA1G, LOX, SLC30A10, ELMO1, HAND1, IBN2 and THBD, and which is capable of amplifying the region comprising the CpG region.

15. The kit according to claim 14, wherein the marker genes are the 24 genes shown in Table 8.
